(19) United States
(12) Patent Application Publication
(10) Pub. No.: US 2012/0251514 A1
(43) Pub. Date: Oct. 4, 2012

(54) MODULATED PROGRAMMED DEATH LIGAND-1

(75) Inventors: Daniel Fowler, Bethesda, MD (US); Shoba Amarnath, North Bethesda, MD (US); Jeffrey Medin, Toronto (CA); James Chian Ming Wang, Scarborough (CA)

(73) Assignees: University Health Network, Toronto, ON (CA); The United States of America, as rep. by the Sec. Department of Health and Human Services, Bethesda, MD (US)

(21) Appl. No.: 13/509,476

(22) PCT Filed: Nov. 12, 2010
(86) PCT No.: PCT/US2010/056450

§ 371 Date: Jun. 19, 2012

Related U.S. Application Data
(60) Provisional application No. 61/261,081, filed on Nov. 13, 2009.

(51) Int. Cl. A61K 31/7088; C12N 15/867; C12N 5/10; C12N 1/15; A61P 37/06; C12N 1/21; C12N 1/11; C12N 15/62; A61K 35/14; C12N 15/63; C12N 1/13

(52) U.S. Cl. 424/93.71; 435/320.1; 435/419; 435/254.11; 435/257.2; 435/252.3; 435/258.1; 435/366; 435/252.33; 435/361; 435/365; 435/369; 435/325; 435/372; 435/372.3; 536/23.2; 514/44 R

(57) ABSTRACT

The invention provides nucleic acids comprising a nucleotide sequence encoding programmed death ligand-1 (PD-L1) and a nucleotide sequence encoding a fusion protein comprising thymidylate kinase (TMPK) or a variant thereof and a cell surface marker or a variant thereof. Recombinant expression vectors, host cells, populations of cells, pharmaceutical compositions, and kits relating to the nucleic acids are disclosed. Methods of treating or preventing a disease in a host and methods of suppressing an immune system in a host are also disclosed.

### CROSS-REFERENCE TO RELATED APPLICATIONS

This patent application claims the benefit of U.S. Provisional Patent Application No. 61/261,081, filed Nov. 13, 2009, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 97,905 Byte ASCII (Text) file named “707002SeqLis_ST25.txt,” created on Nov. 10, 2010.

### BACKGROUND OF THE INVENTION

Autoimmune diseases involve an inappropriate immune reaction against self cells, tissues, and/or organs. Allo-immune diseases involve an immune reaction against foreign or transplanted organs, tissues, and/or cells as in the case of, organ transplantation, for example. Autoimmune and allo-immune diseases can lead to serious complications and may be chronic, debilitating, and/or fatal. Accordingly, there is a need in the art for compositions and methods useful for suppressing the immune system and for treating autoimmune and allo-immune diseases.

### BRIEF SUMMARY OF THE INVENTION

An embodiment of the invention provides a nucleic acid comprising a nucleotide sequence encoding programmed death ligand-1 (PD-L1) and a nucleotide sequence encoding a fusion protein comprising thymidylate kinase (TMPK) or a variant thereof and a cell surface marker or a variant thereof.

Further embodiments of the invention provide related recombinant expression vectors, host cells, populations of cells, pharmaceutical compositions, and kits relating to the nucleic acids of the invention.

Additional embodiments of the invention provide methods of treating or preventing a disease in a host and methods of suppressing an immune response in a host.

### BRIEF DESCRIPTION OF THE SEVERAL VIEWS OF THE DRAWING(S)

FIG. 1 is a graph of flow cytometry detection of the PD-L1 binding partners PD1 and CD80 on effector CD4+ cells after exposure to control dendritic cells (DC), CD4 conditioned (DC_{CD4}), or Treg conditioned (DC_{Treg}) allogeneic DC for 48 hours (mean±SEM of n=3 experiments).

FIG. 2 is a graph of flow cytometry detection of the PD-L1 binding partners PD1 and CD80 on effector CD8+ cells after exposure to control dendritic cells (DC), CD4 conditioned (DC_{CD4}), or Treg conditioned (DC_{Treg}) allogeneic DC for 48 hours (mean±SEM of n=3 experiments).

FIG. 3 is a graph of percent survival of Rag2⁻/⁻γc⁻/⁻ mice at days following transfer of effector T cells (T_{eff}) in combination with allogeneic DC without (black squares) or with (▾) Treg cells (Treg), control CD4 cell-conditioned DC (DC_{CD4}) (▴), or Treg-conditioned DC (DC_{Treg}) (white diamond) (black square versus ▾: p=0.014; black square versus white diamond: p=0.014).

FIG. 4 is a graph of post-transplant weight loss of Rag2^{-/-}γc⁻/⁻ mice at days following transfer of effector T cells (T_{eff}) in combination with allogeneic DC alone (black squares) or with Treg cells (Treg) (▾) or with control CD4 cells (CD4) (Δ), control CD4 cell-conditioned DC (DC_{CD4}) (▴), or Treg-conditioned DC (DC_{Treg}) (white diamond).

FIG. 5 is a graph of percent survival of Rag2⁻/⁻γc⁻/⁻ mice at days following transfer of effector T cells (T_{eff}) in combination with Treg-conditioned DC (DC_{Treg}) that were incubated with anti-PD-L1 (αPD-L1) (▾), isotype control antibody (▴), or no antibody (white diamond) 30 minutes prior to adoptive transfer (white diamond versus ▾: p=0.015; ▾ versus ▴: p=0.015).

### DETAILED DESCRIPTION OF THE INVENTION

Programmed death (PD) ligand 1 (PD-L1, also known as B7-H1) is an immune inhibitory molecule. PD-L1 is expressed on DC, human tumor cells, and normal human tissue and interacts with PD receptors on T cells to modulate the balance of tolerance and immunity. In humans, intra-tumor T regulatory cells (Tregs) inhibit responder T cell proliferation through PD-L1. In accordance with an embodiment of the invention, it has been discovered that human Treg cells modulate dendritic cells (DCs) by upregulating PD-L1 expression by DCs. The modulated DCs, in turn, suppress T cells through a PD-L1 dependent mechanism. Treg cells are not plentiful in the peripheral circulation and are not easily expanded ex vivo. Without being bound to a particular theory, it is believed that cells expressing the inventive nucleic acid exhibit the immunosuppressant characteristics of Treg cells. By expressing the inventive nucleic acids in host cells that are easily obtained, e.g., activated T cells, the inventive nucleic acids advantageously ameliorate the difficulties encountered in isolating and expanding Treg cells. Advantageously, it has also been discovered that a nucleic acid comprising a nucleotide sequence encoding PD-L1 and a nucleotide sequence encoding a fusion protein comprising TMPK or a variant thereof and either cluster of differentiation 19 (CD19) or CD34 or variants thereof can provide PD-L1 as a therapeutic molecule in combination with the ability to specifically eliminate cells expressing the nucleotide sequence after the therapeutic effect has been realized. In accordance with an embodiment of the invention, host cells expressing the inventive nucleic acids can provide a therapeutic composition for adoptive cell transfer therapy to treat or prevent disease and/or suppress an immune response in a host.

Thus, an embodiment of the invention provides a nucleic acid comprising a nucleotide sequence encoding PD-L1 and a nucleotide sequence encoding a fusion protein comprising TMPK or a variant thereof and a cell surface marker such as, e.g., CD19 or CD34 or variants thereof.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

In an embodiment of the invention, the nucleic acid comprises a nucleotide sequence encoding PD-L1. Accordingly, adoptive transfer of host cells expressing the inventive nucleic acid provide a source of PD-L1 that will be capable of reducing or eliminating auto-immune or allo-immune pathogenic cells that express PD-1. Prior to their therapeutic application, host cells expressing PD-L1 may be tracked, selected, sorted, and/or purified using any suitable method, e.g., flow cytometry. The PD-L1 may be any mammalian PD-L1. Preferably, the PD-L1 is human PD-L1 (SEQ ID NO: 1). The nucleotide sequence encoding PD-L1 can comprise the wild-type, naturally-occurring nucleotide coding sequence of PD-L1. In this regard, the nucleotide sequence encoding PD-L1 can comprise, consist, or consist essentially of SEQ ID NO: 2 (wild-type PD-L1).

Alternatively, the nucleotide sequence encoding PD-L1 can be a sequence that has undergone codon optimization. Without being bound to a particular theory, it is believed that optimization of the nucleic acid sequence increases the translation efficiency of the mRNA transcripts. Optimization of the nucleic acid sequence may involve substituting a native codon for another codon that encodes the same amino acid, but can be translated by tRNA that is more readily available within a cell, thus increasing translation efficiency. Optimization of the nucleic acid sequence may also reduce secondary mRNA structures that would interfere with translation, thus increasing translation efficiency. In this regard, the nucleotide sequence encoding PD-L1 can comprise, consist, or consist essentially of, SEQ ID NO: 3 (codon-optimized PD-L1).

In an embodiment of the invention, the nucleic acid also comprises a nucleotide sequence that encodes TMPK or a variant thereof. TMPK catalyzes the addition of a phosphoryl group to a prodrug such as, e.g., thymidylate and thymidine analogs such azidothymidine (AZT). Phosphorylation of the prodrug activates the prodrug so that it specifically kills cells that express the inventive nucleotide sequence. Accordingly, delivery of PD-L1 can be halted upon administration of AZT. Thus, the inventive nucleotide sequence advantageously provides a “safety switch,” i.e., the capability of halting delivery of PD-L1, and thus the switch allows the duration of exposure to PD-L1 to be controlled. The nucleotide sequence that encodes TMPK may be, for example, any of those disclosed in WO 2008/116316, WO 2009/114942 (set forth herein as nucleotide SEQ ID NOs: 13, 15, 17, 19, and 21 encoding amino acid SEQ ID NOs: 14, 16, 18, 20, and 22, respectively), U.S. Patent Application Publication Nos. 2009/0074733 and 2009/0068158, or GenBank Accession Nos. NM 012145, X54729, AY893951, BT020055, or L16991. In one embodiment, the TMPK variant may include substitutions, insertions, or deletions (e.g., truncations), wherein the TMPK variant increases phosphorylation of the prodrug relative to phosphorylation of the prodrug by wild-type (unmodified) TMPK. Exemplary nucleotide sequences encoding a TMPK variant are disclosed in WO 2009/114942 (set forth herein as SEQ ID NOs: 25, 28, 29, and 37 encoding amino acid SEQ ID NOs: 26, 23, 24, and 39, respectively), WO 2008/116316 and in U.S. Patent Application Publication Nos. 2009/0074733 and 2009/0068158. In a preferred embodiment, as discussed in more detail below, the nucleotide sequence encodes TMPK or a variant thereof fused to a cell surface marker or a variant thereof.

In an embodiment of the invention, the nucleic acid also comprises a nucleotide sequence that encodes a cell surface marker or a variant thereof. In one embodiment, the cell surface marker variant may include substitutions, insertions, or deletions (e.g., truncations) as compared to the wild-type (unmodified) cell surface marker. Exemplary cell surface markers include CD19 (Genbank Accession No. M84371), truncated CD19, CD25 (Genbank Accession No. NM_{—}000417; set forth herein as SEQ ID NO: 46), low affinity nerve growth factor receptor (LNGFR) (Genbank Accession No. NM_{—}002507; set forth herein as SEQ ID NO: 47), truncated LNGFR, CD34 (Genbank Accession No. AB238231), truncated CD34, erythropoietin receptor (EpoR), HSA and CD20 (Genbank Accession No. NM_{—}152866; set forth herein as SEQ ID NO: 45). The cell surface marker allows cells expressing the inventive nucleotide sequence to be tracked, selected, sorted, and/or purified, thereby advantageously providing a purified cell product. In a preferred embodiment, the cell surface marker is CD19, truncated CD19, CD34, or truncated CD34. Exemplary nucleotide sequences encoding CD19 (including truncated CD19) are disclosed in WO 2009/114942 (set forth herein as SEQ ID NOs: 30, 33, and 38), WO 2008/116316 and in U.S. Patent Application Publication Nos. 2009/0074733 and 2009/0068158. In an embodiment, the nucleotide sequence encoding CD19 or truncated CD19 encodes amino acid SEQ ID NOs: 31, 32, 34, and 40. In a preferred embodiment, as discussed in more detail below, the nucleotide sequence encodes a cell surface marker (or a variant thereof) fused to TMPK (or a variant thereof).

In a particularly preferred embodiment of the invention, the nucleic acid comprises a nucleotide sequence encoding a fusion protein comprising any of the TMPK nucleotide sequences described herein and any of the nucleotide sequences encoding a cell surface marker described herein. The TMPK (or a variant thereof) and the cell surface marker (or a variant thereof) are fused in frame such that both components are expressed together as one continuous polypeptide sequence.

In an embodiment, the nucleotide sequence encoding a fusion protein comprises a linker sequence that encodes residues that link the TMPK (or a variant thereof) and cell surface markers (or a variant thereof), as disclosed in WO 2009/114942 (set forth herein as SEQ ID NO: 35, encoding SEQ ID NO: 36). The linker, when referring to an amino acid sequence that links the TMPK (or a variant thereof) and cell surface markers (or a variant thereof), optionally comprises about 3, about 4, about 5, about 6, about 6 to about 10, about 10 to about 15 or about 15 to about 25 amino acids or longer and when referring to a nucleotide sequence that links the TMPK (or a variant thereof) and cell surface markers (or a variant thereof) comprises about 3 to about 6, about 6 to about 12, about 18, about 12 to about 24, or about 24 to about 72 nucleic acid residues or longer. The fusion protein may comprise a cell surface marker (or variant thereof) fused to a linker, which in turn is fused to TMPK (or a variant thereof) in any order (e.g., NH₂-TMPK-linker-cell-surface marker-COOH, NH₂-cell surface marker-linker-TMPK-COOH, 5′-TMPK-linker-cell surface marker-3′,5′-cell surface marker-linker-TMPK-3′). Exemplary nucleotide sequences encoding a fusion protein comprising CD19 and TMPK (or variants thereof) are disclosed in WO 2009/114942 (set forth herein as SEQ ID NO: 42, encoding SEQ ID NO: 41; and SEQ ID NO: 43, encoding SEQ ID NO: 44). In this respect, the fusion protein can comprise, consist, or consist essentially of, an amino acid sequence encoding a variant of TMPK and truncated CD19 (SEQ ID NO: 4). In this regard, the nucleotide sequence encoding a fusion protein comprising a variant of TMPK and truncated CD19 comprises, consists of, or consists essentially of SEQ ID NO: 5.

The nucleotide sequence encoding a fusion protein comprising TMPK (or a variant thereof) and a cell surface marker (or a variant thereof) advantageously expresses TMPK (or a variant thereof) and the cell surface marker (or a variant thereof) in a ratio of about 0.5 to about 1.5, preferably about 0.8 to about 1.2, most preferably about 1 to about 1. Accordingly, the inventive nucleic acid makes it possible to track, select, sort, and/or purify on the basis of the cell surface marker and also simultaneously track, select, sort, and/or purify those cells that also express the TMPK “safety switch.” Therefore, prior to their therapeutic application in host cells, the inventive nucleic acids advantageously make it possible to eliminate or reduce the number of cells that do not express the TMPK “safety switch.” Accordingly, the host may be protected against any pathologic effect that might be mediated by the host cells expressing the inventive nucleic acid. That is, when it is desirable to halt exposure to the cells expressing the inventive nucleic acid (e.g., when a therapeutic effect has been realized), a prodrug (e.g., AZT) may be administered to the host. The prodrug will be activated (e.g., phosphorylated) in the host cells expressing the inventive nucleic acid, thereby resulting in clonal elimination of the host cells expressing the inventive nucleic acid.

In an embodiment, the nucleic acid comprises a nucleotide sequence encoding programmed death ligand-1 (PD-L1) that encodes SEQ ID NO: 1 and a nucleotide sequence encoding a fusion protein comprising thymidylate kinase (TMPK) or a variant thereof and CD19 or a variant thereof that encodes SEQ ID NO: 4.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive nucleic acids. It is generally appreciated that conditions can be rendered more stringent by the addition of increasing amounts of formamide.

The nucleic acids of the invention can be incorporated into a recombinant expression vector. In this regard, an embodiment of the invention provides recombinant expression vectors comprising any of the nucleotide sequences of the invention. In an embodiment of the invention, the recombinant expression vector comprises a nucleotide sequence encoding PD-L1 and a TMPK variant fused to truncated CD19 (SEQ ID NO: 6). For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transduce any suitable host. Suitable vectors include those designed for propagation and expansion or for expression or both, such as plasmids and viruses. The vector can be selected from the group consisting of the pUC series (Feunentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), 2 EMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector or a lentiviral vector. In this regard, the recombinant expression vector comprises, consists, or consists essentially of, SEQ ID NO: 7.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In a preferred embodiment, the recombinant expression vector is a lentiviral vector comprising woodchuck hepatitis virus post-transcriptional regulation element (WPRE) (SEQ ID NO: 27).

The recombinant expression vector can include one or more marker genes, which allow for selection of transformed or transduced hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter operably linked to the inventive nucleotide sequence, or to the nucleotide sequence which is complementary to or which hybridizes to the inventive nucleotide sequence. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus. In a preferred embodiment, the recombinant expression vector comprises an elongation factor-1 alpha (EF1-alpha) promoter (SEQ ID NO: 8).

Another embodiment of the invention further provides a host cell comprising any of the recombinant expression vectors described herein. As used herein, the term “host cell” refers to any type of cell that can contain the inventive recombinant expression vector. The host cell can be an artificial cell such as, e.g., a liposome. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant PD-L1, the host cell is preferably a mammalian cell. One of ordinary skill in the art may, for example, select a host cell that expresses one or more cytokines or chemokine receptors that may home the host cell to a particular part of the body or may be otherwise advantageous for the particular disease being treated. Most preferably, the host cell is a human cell. While the host cell can be of any cell type, can originate from any type of tissue, and can be of any developmental stage, the host cell preferably is a peripheral blood mononuclear cell (PBMC) or a peripheral blood leukocyte (PBL). More preferably, the host cell is a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. More preferably, the T cell is a T cell isolated from a human. The T cell can be any type of T cell and can be of any developmental stage, including but not limited to, CD4⁺/CD8⁺ double positive T cells, CD4⁺ helper T cells, e.g., cytotoxic T cells (Tc1 or Tc2), Th₁ and Th₂ cells, CD8⁺ T cells (e.g., cytotoxic T cells), tumor infiltrating cells (TILs), memory T cells, naïve T cells, and the like. Preferably, the T cell is a CD8⁺ T cell or a CD4⁺ T cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

In a preferred embodiment of the invention, the cell or population of cells may be cultured in the immunosuppressant agent rapamycin, as described in U.S. Patent Application Publication No. 2006/0159667 to produce rapamycin-resistant cells. Cell(s) cultured in rapamycin may, advantageously, have an anti-apoptotic phenotype and may be selectively resistant to the inhibitory effects of rapamycin in vivo. Accordingly, in vivo administration of rapamycin-resistant cells with concomitant administration of rapamycin may inhibit non-cultured cells that may not possess the inventive nucleic acid and at the same time allow preferential expansion of the in vitro cultured cell comprising the inventive nucleic acid. Cell(s) with an anti-apoptotic phenotype may, advantageously, improve host cell persistence in vivo after adoptive transfer.

The inventive nucleic acids, recombinant expression vectors, and host cells (including populations thereof) can be isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70% or 80%, or can be 100%.

The inventive nucleic acids, recombinant expression vectors, and host cells (including populations thereof), all of which are collectively referred to as “inventive PD-L1 materials” hereinafter, can be formulated into a composition, such as a pharmaceutical composition. In this regard, an embodiment of the invention provides a pharmaceutical composition comprising any of the nucleic acids, expression vectors, and host cells (including populations thereof), and a pharmaceutically acceptable carrier. The inventive pharmaceutical compositions containing any of the inventive PD-L1 materials can comprise more than one inventive PD-L1 material, e.g., a nucleic acid and a host cell, or two or more different nucleic acids. Alternatively, the pharmaceutical composition can comprise an inventive PD-L1 material in combination with another pharmaceutically active agent or drug, such as an immunosuppressant agent. In one embodiment, the pharmaceutical composition can comprise an inventive PD-L1 material in combination with rapamycin.

Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive PD-L1 material, as well as by the particular method used to administer the inventive PD-L1 material. Accordingly, there are a variety of suitable formulations of the pharmaceutical composition of the invention. The following formulations for parenteral, intratumoral, subcutaneous, intravenous, intramuscular, intraarterial, intrathecal, and interperitoneal administration are exemplary and are in no way limiting. More than one route can be used to administer the inventive PD-L1 materials, and in certain instances, a particular route can provide a more immediate and more effective response than another route.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inventive PD-L1 material can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the inventive PD-L1 material in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)). Preferably, when administering cells, e.g., T cells, the cells are administered via injection.

It will be appreciated by one of skill in the art that, in addition to the above-described pharmaceutical compositions, the inventive PD-L1 materials of the invention can be formulated as inclusion complexes, such as cyclodextrin inclusion complexes, or liposomes.

For purposes of the invention, the amount or dose of the inventive PD-L1 material administered should be sufficient to effect, e.g., a therapeutic or prophylactic response, in the subject or animal over a reasonable time frame. For example, the dose of the inventive PD-L1 material should be sufficient to suppress an immune response in the host. Suppression of the immune response can be characterized by, for example, the inhibition of T cell proliferation or activation as measured by, for example, a decreased CD4+ and CD8+ cell count in the peripheral blood by, e.g., flow cytometry or decreased serum levels of inflammatory cytokines such as, e.g., IL-1-alpha or TNF-alpha. With respect to a human host, it is believed that suppression of the immune response may occur in a period of from about 2 hours or longer, e.g., 12 to 24 or more hours, from the time of administration. The suppression of the immune response may be durable for at least an extended period of time. In certain embodiments, the time period for initial onset of suppression of the immune response could be even longer. The dose will be determined by the efficacy of the particular inventive PD-L1 material and the condition of the mammal (e.g., human), as well as the body weight of the mammal (e.g., human) to be treated.

Many assays for determining an administered dose are known in the art. For purposes of the invention, a dose of host cells expressing the inventive nucleic acids may range, for example, from about 5×10⁶ cells per kg of recipient body weight to about 25×10⁶ cells per kg of recipient body weight. This dose range can, advantageously, be readily achieved in a clinical scale of T cell manufacturing, e.g., in the manufacture of rapamycin-resistant T cells.

The dose of the inventive PD-L1 material also will be determined by the existence, nature and extent of adverse side effects that might accompany the administration of a particular inventive PD-L1 material. Typically, the attending physician will decide the dosage of the inventive PD-L1 material with which to treat each individual patient, taking into consideration a variety of factors, such as age, body weight, general health, diet, sex, inventive PD-L1 material to be administered, route of administration, and the severity of the condition being treated. By way of example and not intending to limit the invention, the dose of the inventive PD-L1 material can be about 0.001 to about 1000 mg/kg body weight of the subject being treated/day, from about 0.01 to about 10 mg/kg body weight/day, about 0.01 mg to about 1 mg/kg body weight/day.

One of ordinary skill in the art will readily appreciate that the inventive PD-L1 materials of the invention can be modified in any number of ways, such that the therapeutic or prophylactic efficacy of the inventive PD-L1 materials is increased through the modification. For instance, the inventive PD-L1 materials can be conjugated either directly or indirectly through a linker to a targeting moiety. The practice of conjugating compounds, e.g., inventive PD-L1 materials, to targeting moieties is known in the art. See, for instance, Wadwa et al., J. Drug Targeting 3: 111 (1995) and U.S. Pat. No. 5,087,616. The term “targeting moiety” as used herein, refers to any molecule or agent that specifically recognizes and binds to a cell-surface receptor, such that the targeting moiety directs the delivery of the inventive PD-L1 materials to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). The term “linker” when referring to the conjugation of the inventive PD-L1 materials to a targeting moiety, refers to any agent or molecule that bridges the inventive PD-L1 materials to the targeting moiety. One of ordinary skill in the art recognizes that sites on the inventive PD-L1 materials, which are not necessary for the function of the inventive PD-L1 materials, are ideal sites for attaching a linker and/or a targeting moiety, provided that the linker and/or targeting moiety, once attached to the inventive PD-L1 materials, do(es) not interfere with the function of the inventive PD-L1 materials, i.e., the ability to suppress an immune response, or to treat or prevent disease.

Alternatively, the inventive PD-L1 materials can be modified into a depot form, such that the manner in which the inventive PD-L1 materials is released into the body to which it is administered is controlled with respect to time and location within the body (see, for example, U.S. Pat. No. 4,450,150). Depot forms of inventive PD-L1 materials can be, for example, an implantable composition comprising the inventive PD-L1 materials and a porous or non-porous material, such as a polymer, wherein the inventive PD-L1 materials is encapsulated by or diffused throughout the material and/or degradation of the non-porous material. The depot is then implanted into the desired location within the body and the inventive PD-L1 materials are released from the implant at a predetermined rate.

It is contemplated that the inventive pharmaceutical compositions, nucleic acids, recombinant expression vectors, host cells, or populations of cells can be used in methods of suppressing an immune response in a host. In this regard, an embodiment of the invention provides a method of suppressing an immune response in a host, comprising administering to the host any of the pharmaceutical compositions, nucleic acids, recombinant expression vectors, host cells, or populations of cells described herein in an amount effective to suppress the immune response in the host.

It is also contemplated that the inventive pharmaceutical compositions, nucleic acids, recombinant expression vectors, host cells, or populations of cells can be used in methods of treating or preventing a disease. In this regard, an embodiment of the invention provides a method of treating or preventing a disease in a host, comprising administering to the host any of the pharmaceutical compositions, nucleic acids, recombinant expression vectors, host cells, or populations of cells described herein in an amount effective to treat or prevent a disease in the host.

The disease that may be treated by the inventive methods may be an autoimmune disease or an allo-immune disease. The autoimmune diseases that may be treated by the inventive methods may be generally characterized by an inappropriate immune reaction against self cells, tissues, and/or organs. Exemplary autoimmune diseases include, but are not limited to: vitiligo, alopecia, autoimmune kidney disease, celiac disease, inflammatory bowel disease, hepatitis, Addison's disease, Hashimoto's disease, Graves disease, atrophic gastritis/pernicious anemia, acquired hypogonadism/infertility, hypoparathyroidism, multiple sclerosis, Myasthenia gravis, Coombs positive hemolytic anemia, systemic lupus erythematosis, allergic diseases (such as asthma, hay fever, or allergic rhinitis), Sjogren's syndrome, rheumatoid arthritis, auto-immune thyroiditis, Crohn's disease, ulcerative colitis, cardiovascular disease (e.g., atherosclerosis), and immune mediated (type-1) diabetes mellitus. The allo-immune diseases that may be treated by the inventive methods may be generally characterized by an immune reaction against foreign or transplanted organs, tissues, and/or cells. Exemplary allo-immune diseases include, but are not limited to: acute and chromic graft-versus-host disease (GVHD) and/or graft rejection that can occur, e.g., in the setting of solid organ transplantation (e.g., pancreatic, renal, cardiac, stem cell, or liver transplantation) and graft-versus-host disease that can occur in the setting of allogeneic hematopoietic stem cell transplantation (e.g., bone marrow, peripheral blood, or cord blood transplantation).

The inventive methods described herein may further comprise administering to the host a prodrug. A prodrug refers to a pharmacological substance (drug) which is administered in an inactive form (or significantly less active form, e.g., at least 90% or at least 95% less active than the active drug form). In one embodiment, the prodrug is a prodrug that is phosphorylated by TMPK (or a variant thereof), activating the prodrug. The activated prodrug specifically kills the cells that express the inventive nucleotide sequence. Accordingly, administering the prodrug to the host halts the delivery of PD-L1 to the host. The prodrug may be administered to the host at any time following administration of the PD-L1 materials to the host. In an embodiment of the invention, the prodrug is administered to the host when the inventive PD-L1 materials ceases to provide a benefit or therapeutic effect to the host. Typically, the attending physician will decide when to administer the prodrug.

In a preferred embodiment, the prodrug is AZT. Without being bound to a particular theory, it is believed that TMPK (or a variant thereof) catalyzes the phosphorylation of AZT-monophosphate to AZT-diphosphate, thereby increasing the intracellular concentration of AZT-triphosphate, which is toxic to the cell. In an alternate embodiment, the prodrug is a thymidine analog that is a substrate for the TMPK (or a variant thereof) of the inventive nucleic acids. In another embodiment the prodrug is a uracil analog.

For purposes of the inventive methods, wherein host cells or populations of cells are administered, the cells can be cells that are allogeneic or autologous to the host. Preferably, the cells are autologous to the host.

While the inventive PD-L1 materials and the inventive methods may be useful for suppressing those cells (e.g., T cells) that mediate auto- and/or allo-immune disease, it may also be useful to provide cells (e.g., T cells) capable of mediating an immune response against detrimental antigens (e.g., tumor antigens, bacterial antigens, viral antigens, etc.) in order to treat or prevent infection and/or cancer in the host while also suppressing the immune response mediating the auto- or allo-immune disease. In this regard, the inventive methods may further comprise administering cells (e.g., T cells) resistant to the immuno-suppressive (e.g., tolerizing) effects of PD-L1 that also have antigenic specificity for detrimental antigens. Without being bound to a particular theory, it is believed that cells expressing the inventive nucleic acid suppress the immune response via the Src homology region 2 domain-containing phosphatase (SHP)-1 and SHP-2 signaling pathway, and that inhibition (e.g., ex vivo inhibition) of the SHP1/2 pathway generates cells resistant to the immunosuppressive (e.g., tolerizing) effect of PD-L1.

In this regard, the method may further comprise treating at least one cell with an SHP1/2 inhibitor ex vivo and administering the SHP1/2 inhibitor-treated cell(s) to the host. The SHP1/2 inhibitor-treated cell may be any type of cell that is described herein for the inventive host cells comprising the inventive nucleic acid and inventive recombinant expression vector. Preferably, the SHP1/2 inhibitor-treated cell is a T-cell. Preferably, the SHP1/2 inhibitor-treated cell lacks the inventive nucleic acid and inventive recombinant expression vector. The inventive method may comprise administering the SHP1/2 inhibitor-treated cell(s) to the host before, during, or after administering any of the inventive PD-L1 materials to the host. Similarly, the inventive method may comprise administering the inventive PD-L1 materials to the host before, during, or after administering the SHP1/2 inhibitor-treated cell(s) to the host.

The SHP1/2 inhibitor may be any suitable SHP1/2 inhibitor. Exemplary SHP1/2 inhibitors include pervanadate and NSC87877.

The SHP1/2 inhibitor-treated cell(s) may have antigenic specificity for any bacterial antigen, viral antigen, tumor antigen, fungal antigen, or parasitic antigen. The phrase “antigenic specificity” as used herein means that the SHP1/2 inhibitor-treated cell(s) can specifically bind to, immunologically recognize, and/or mediate an immune response against a bacterial antigen, a viral antigen, a tumor antigen, a fungal antigen, or a parasitic antigen. An immune response may be characterized by an increased production of cytokines such as, e.g., interferon gamma (IFNγ), the stimulation of a cell-mediated immune response such as, e.g., the activation of T-cells and/or macrophages, and/or the destruction of cells expressing the bacterial antigen, viral antigen, tumor antigen, fungal antigen, or parasitic antigen.

The term “tumor antigen” as used herein refers to any molecule (e.g., protein, peptide, lipid, carbohydrate, etc.) solely or predominantly expressed or over-expressed by a tumor cell or cancer cell, such that the antigen is associated with the tumor or cancer. The tumor antigen can additionally be expressed by normal, non-tumor, or non-cancerous cells. However, in such cases, the expression of the tumor antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the tumor antigen can additionally be expressed by cells of a different state of development or maturation. For instance, the tumor antigen can be additionally expressed by cells of the embryonic or fetal stage, which cells are not normally found in an adult host. Alternatively, the tumor antigen can be additionally expressed by stem cells or precursor cells, which cells are not normally found in an adult host.

The tumor antigen can be an antigen expressed by any cell of any cancer or tumor. For example, the tumor antigen can be an antigen expressed by any cell of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, uterine cancer, esophageal cancer, cervical cancer, gastrointestinal carcinoid tumor, lymphoid and other hematopoietic tumors, Hodgkin lymphoma, B cell lymphoma, bronchial squamous cell cancer, hypopharynx cancer, kidney cancer, larynx cancer, liver cancer, pancreatic cancer, carcinoma, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. Examples of tumor antigens include proteins such as Ig-idiotype of B cell lymphoma, mutant cyclin-dependent kinase 4 of melanoma, Pmel-17 (gp100) of melanoma, MART-1 (Melan-A) of melanoma, p15 protein of melanoma, tyrosinase of melanoma, MAGE 1, 2 and 3 of melanoma, thyroid medullary, small cell lung cancer, colon and/or bronchial squamous cell cancer, BAGE of bladder, melanoma, breast, and squamous-cell carcinoma, gp75 of melanoma, oncofetal antigen of melanoma; carbohydrate/lipids such as muci mucin of breast, pancreas, and ovarian cancer, GM2 and GD2 gangliosides of melanoma; oncogenes such as mutant p53 of carcinoma, mutant ras of colon cancer and HER21neu proto-onco-gene of breast carcinoma; viral products such as human papilloma virus proteins of squamous cell cancers of cervix and esophagus.

The viral antigen can be an antigen expressed by any virus. For example, the viral antigen can be an antigen expressed by any of the following viruses: adenovirus, chicken pox, cytomegalovirus, dengue, hepatitis A, hepatitis B, hepatitis C, human immune deficiency virus (HIV), herpes simplex virus (HSV)-1, HSV-2, influenza, Japanese encephalitis, measles, mumps, papilloma virus, papova virus, polio, rabies, respiratory syncytial virus, rhinovirus, rubella, severe acute respiratory syndrome (SARS), rotavirus, smallpox, yellow fever, echinovirus, arbovirus, hantavirus, coxsackie virus, echovirus, picornaviridae, enteroviruses, caliciviridae, alphaviruses, flaviviruses, coronaviruses, Marburg viruses, ebola viruses, parainfluenza virus, orthomyxoviruses, bunyaviruses, arenaviruses, reoviruses, orbiviruses, human T cell leukemia virus type I, human T cell leukemia virus type II, lentiviruses, varicella-zoster virus (VZV), polyomaviruses, parvoviruses, and Epstein Barr virus. Examples of viral antigens include surface antigen gD or gB of herpes simplex virus (HSV) type 1 or type 2; surface antigen gpI or gpIII of varicella-zoster virus (VZV); gag antigen, Nef, p24, gp120, gp41, Tat, Rev, Pol, or env antigen of human immunodeficiency virus (HIV); gag antigen or env antigen of adult human T cell leukemia virus (HTLV-I); C antigen, M antigen or E antigen of hepatitis C virus (HCV); VP4 and VP7 of rotavirus; hemagglutinin, neuraminidase surface protein, and nucleoprotein of influenza; thymidine kinase of herpes simplex virus; IPV or OPV of polio virus; lyophilized inactivated virus of rabies; and core antigen, surface antigen L protein, surface antigen M protein or surface antigen S protein of hepatitis B virus (HBV).

The bacterial antigen can be an antigen expressed by any bacteria. For example, the bacterial antigen can be an antigen expressed by any of the following bacteria: Borellia burgdorferi, Neisseria meningitidis, Streptococcus pneumoniae, Neisseria gonorrhoeae, Chlamydia pneumoniae, Chlamydia trachomatis, Bordetella pertussis, Helicobacter pylori, Corynebacterium diphtheriae, Clostridium tetani, Escherichia coli, Haemophilus influenzae B, Legionella pneumoniae, Porphyromonas gingivalis, Moraxella catarrhalis, Streptococcus agalactiae, Streptococcus pyogenes, Staphylococcus aureus, Mycobacterium, Salmonella, Shigella, Rickettsia, Listeria, Pseudomonas, and Vibrio. Examples of bacterial antigens include protein antigen from N. meningitides serogroup B; saccharide antigen from N. meningitides serogroup A, C, W135, and/or Y, such as the oligosaccharide from serogroup C; a saccharide antigen from S. pneumoniae; pertussis holotoxin (PT), pertactin, agglutinogens 2 and 3, adenylate cyclase-hemolysin, and filamentous haemagglutinin (FHA) of B. pertussis; diphtheria toxoid, e.g., the CRM₁₉₇ mutant of diptheria; tetanus toxoid; CagA, VacA, NAP, HopX, HopY and urease of H. pylori; a saccharide antigen from H. influenzae B; Shigella sonnei form 1 antigen; O-antigen of V. cholerae Inaba strain 569B; CFA/I fimbrial antigen and nontoxic B-subunit of the heat-labile toxin of E. coli; and fragment C of tetanus toxin of C. tetani.

The parasitic antigen can be an antigen expressed by any parasite. For example, the parasitic antigen can be an antigen expressed by any of the following parasites: Plasmodium, Trypanosoma, Giardia, Boophilus, Babesia, Entamoeba, Eimeria, Fasciola, Leishmania, Schistosome, Brugia, Fascida, Dirofilaria, Trichostrongylus, Wuchereria, and Onchocerea. Examples of parasitic antigens include circumsporozoite antigens of Plasmodium, such as the circumsporozoite antigen of P. bergerii or the circumsporozoite antigen of P. falciparum; the merozoite surface antigen of Plasmodium; the galactose specific lectin of E. histolytica; gp63 of Leishmania; paramyosin of B. malayi, the triose-phosphate isomerase of S. mansoni; the secreted globin-like protein of T. colubriformis; the glutathione-S-transferases of F. hepatica; S. bovis and S. japonicum; and KLH of S. bovis and S. japonicum.

The fungal antigen can be an antigen expressed by any fungus. For example, the fungal antigen can be an antigen expressed by any of the following fungi: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, Trichophyton faviforme, Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia, Fonsecaea, Wangiella, Sporothrix, Basidiobolus, Conidiobolus, Rhizopus, Mucor, Absidia, Mortierella, Cunninghamella, Saksenaea, Alternaria, Curvularia, Helminthosporium, Fusarium, Aspergillus, Penicillium, Monolinia, Rhizoctonia, Paecilomyces, Pithomyces, and Cladosporium. Examples of fungal antigens include heat shock protein 60 (HSP60) of Histoplasma; capsular polysaccharides of Cryptococcus; spherule antigens of Coccidioides; and tinea fungal antigens such as trichophytin of Coccidioides.

An embodiment of the invention provides a kit comprising any of the PD-L1 materials described herein and a prodrug. In this regard, an embodiment of the invention provides any of the pharmaceutical compositions, nucleic acids, recombinant expression vectors, host cells, or populations of cells described herein in combination with a prodrug. The prodrug may be any of the prodrugs described herein. In a preferred embodiment, the prodrug is AZT.

In an embodiment, the kit may further comprise at least one cell. The cell may lack the inventive nucleic acid and the inventive recombinant expression vector. The cell may be a SHP1/2 inhibitor-treated cell, as described above. The cell may be any type of cell described for the inventive host cell comprising the inventive nucleic acid and the inventive recombinant expression vector. Preferably, the cell is a T cell. In an embodiment of the invention, the cell may have antigenic specificity for any bacterial antigen, viral antigen, tumor antigen, fungal antigen, or parasitic antigen described above.

In an embodiment of the invention, the kit may further comprise a SHP1/2 inhibitor. The SHP1/2 inhibitor may be any suitable inhibitor. Preferably, the SHP1/2 inhibitor is pervanadate or NSC87877.

The host referred to in the inventive methods can be any host. Preferably, the host is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a disease in a mammal. Furthermore, the treatment or prevention provided by the inventive method can include treatment or prevention of one or more conditions or symptoms of the disease, e.g., autoimmune or allo-immune disease, being treated or prevented. Also, for purposes herein, “prevention” can encompass delaying the onset of the disease, or a symptom or condition thereof.

The following examples further illustrate the invention but, of course, should not be construed as in any way limiting its scope.

### EXAMPLES

### Antibodies and Reagents

X-VIVO 20 media was obtained from BioWhitaker (Walkersville, Md.) and AB serum was from Gem Cell (West Sacramento, Calif.). Anti-CD3, anti-CD28 coated tosyl-activated magnetic beads were provided by Dr. Bruce Levine, University of Pennsylvania. Rapamycin was from Wyeth (Rapamune®; Philadelphia, Pa.). Recombinant human (rh) IL-2 was from PeproTech (Rocky Hill, N.J.); rhIFN-α was from Schering Plough (Kenilworth, N.J.). Recombinant human (rh) rhIL-12 was from PeproTech (Rocky Hill, N.J.) and rhTGF-β1, αTGF-β1, -β2, -β3, and purified αPD-L1 were from R&D Systems (R&D; Minneapolis, Minn.). All antibodies for flow cytometry were obtained from BD Biosciences (San Diego, Calif.) unless otherwise stated. CD4 microbeads were from Miltenyi Biotec (Auburn, Calif.). Sheep anti-mouse (SAM) IgG dynabeads were from Dynal (Carlsbad, Calif.). Anti-human Foxp3 APC was from eBioscience (San Diego, Calif.). Luminex kits for detection of IFN-γ and TNF-a were from Bio-Rad (Hercules, Calif.). 5-(and -6)-carboxyfluorescein diacetate, succinimidyl ester [5(6)-CFDA, SE; CFSE] was from Invitrogen (Carlsbad, Calif.).

### T Cell Subset Isolation

Normal donor peripheral blood cells were collected by apheresis on an IRB-approved protocol. Total lymphocytes were isolated by elutriation (Abrahamsen et al. (1991) J. Clin. Apher. 6(1): 48-53). Total CD4⁺ T cells were then enriched by CD4 microbeads according to manufacturer instructions. To isolate CD127-depleted CD4⁺ T cells: (1) elutriated lymphocytes were adjusted to 100×10⁶ cells/ml and incubated with anti-CD127 (10 μg/ml, 30 minutes, 4° C.); (2) cells were washed, mixed with SAM dynabeads (bead:cell ratio, 4:1), incubated (30 minutes, 4° C.), separated (hand-held magnet, Dynal); and (3) CD127-depleted cells were subjected to CD4 cell isolation by microbeads.

### Ex Vivo Culture of Total CD4⁺ and CD4⁺CD127⁻ T Cell Subsets

Total CD4⁺ and CD4⁺CD127⁻ T cells were co-stimulated and expanded in media containing IL-2, TGF-β1, and rapamycin to generate control bulk “CD4” and “Treg” populations that were directly compared in each experiment.

Total CD4⁺ and CD4⁺CD127⁻ T cells were cultured in polystyrene tissue culture flasks (Corning; Lowell, Mass.). Cells were activated by anti-CD3, anti-CD28 co-stimulation (bead:cell ratio, 3:1) and cultured in X-VIVO 20 with 5% heat-inactivated (HI) AB serum containing rapamycin (1 μM), TGF-β1 (20 ng/ml), rhIL-2 (100 IU/ml). rhIL-2 alone was added at days 2, 4, and 6. Cultures were started at 1.5×10⁶ cells/ml, maintained at 1×10⁶ cells/ml through day 7, and then split daily to 0.5×10⁶/ml by addition of IL-2 and rapamycin-replete media through day 12.

Expanded T cells maintained their CD127⁻ status, were comparable in terms of expansion (bulk CD4 expanded about 70 fold; bulk Treg expanded about 60 fold), co-expression of CD62L with CCR7 (˜65% CD4 CD62L+CCR7+; ˜60% Treg CD62L+CCR7+), and Foxp3 expression (˜75% CD4 Fox3p+; ˜75% Treg Fox3p+). Because Foxp3 is expressed in human Tregs and transiently expressed in human effector T cells (Gavin et al. (2006) PNAS 103(17): 6659-6664), it was reasoned that bulk CD4 cell Foxp3 content may represent a marker of effector differentiation. To address this, ex vivo expanded T cells were compared for simultaneous expression of Foxp3 and effector cytokines, including IL-2 (Foxp3⁺IL-2⁺ events) and IFN-γ (Foxp3+IFN-γ⁺ events). Indeed, relative to Tregs, control CD4 cells had increased co-expression of Foxp3 with IL-2 (CD4˜18% Fox3p+IL2+; Tregs ˜7% Fox3p+IL2+; p=0.030) and Foxp3 with IFN-γ (CD4˜1.4% Fox3p+IFNγ; Tregs ˜0.75% Fox3p+IFNγ; p=0.046). Furthermore, relative to control CD4 cells, expanded Tregs mediated increased suppression of CD4⁺ (bulk CD4˜12% CD4 suppression; Tregs ˜35% CD4 suppression; p=0.017) and CD8⁺ (bulk CD4˜5% CD4 suppression; Tregs ˜30% CD4 suppression; p=0.014) T cell alloreactivity; suppression was observed at a Treg cell to responder T cell ratio of 1:20 that approximates the physiologic ratio.

### Flow Cytometry

T cells were washed with PBS supplemented with 0.1% BSA and 0.01% azide, and stained using anti-: CD4 PE-cy7 (clone S3.5; Caltag; Carlsbad, Calif.), Foxp3 APC (clone 249D; eBioscience), CCR7PE (clone 150503; R&D), CTLA-4 Biotin (clone BN13), CD27 FITC (clone M-T271) and CD62L APC-cy7 (clone DREG-56; Biolegend; San Diego, Calif.). For intra-cellular (IC) flow cytometry, fixation and permeabilization buffer was utilized (eBioscience); four-color IC flow cytometry was performed with combinations of anti-: IL-2 Biotin (clone B33-2), IFN-γ APC (clone B27), CD4 Pe-Cy5 (clone RPA-T4), and Foxp3 PE (clone PCH101; eBioscience). DC were evaluated using anti-: CD80 Bio (clone L307.4), CD86 APC (clone 2331), CD14 PE (clone M5E2), CD83 FITC (clone HB15e), CD40 APC (clone 5C3), and PDL1 PE-cy7 (clone MIH1).

### Generation of Myeloid DC

Monocytes from four healthy, randomly selected donors were obtained by apheresis and elutriation; HLA typing confirmed that the donors did not share major haplotypes. Each monocyte population was cultured in X-VIVO 20 media with 5% HI-AB serum, rhGM-CSF (50 ng/ml), and rhIL-4 (20 ng/ml). On day 5, each DC culture was enumerated and subjected to flow cytometry to document a DC phenotype (CD14⁻, CD11c⁺, CD83⁺, CD80⁺, CD86⁺). The four separate DC populations were pooled in equal proportions and aliquots of the final product were cryopreserved and utilized for each experiment.

### Allogeneic Mixed Lymphocyte Reaction (MLR)

Normal donor lymphocytes (“responder T cells”; 2×10⁵ cells) were co-cultured with allogeneic DCs (5×10⁴ cells) in 96-well round bottom plates (T cell to DC ratio, 20:1). To detect proliferation, responder T cells were CFSE-labeled before co-culture. From the same normal donors, Tregs were generated from CD4⁺CD127⁻ cells or as a control, from total CD4⁺ cells. Initial experiments determined that a Treg to responder T cell ratio of 1:20 consistently yielded suppression of proliferation. Proliferation of CD4⁺ and CD8⁺ responder T cells was evaluated by CFSE dye dilution; percent suppression of CD4 and CD8 responder T cell suppression was calculated, with values representing the ratio of total divided peaks to both divided and non-divided peaks, normalized to the sham-treated experimental group.

### Mechanistic Assays Relating to the MLR

During the MLR, neutralizing antibodies were added, including anti-: CTLA-4, IL-10, TGF-β1, TGF-β2, TGF-β3, LAP, and their respective isotope controls. All antibodies were used at 20 μg/ml. A combination of anti-CTLA-4, anti-TGF-β, and anti-LAP was also tested. 1-methyl-D-tryptophan (1-MT, 1 mM; Sigma) was utilized to inhibit indoleamine 2,3-dioxygenase (IDO). Transwell plates with a 4 mm membrane (Corning LifeSciences; Corning, N.Y.) were utilized to assess Treg cell contact dependency.

### Isolation of Treg-Conditioned Dendritic Cells

For the secondary transfer experiments, DC were incubated with Tregs for 48 hrs (Treg to DC ratio, 1:1). Treg cells were then removed using T cell positive selection (anti-CD3 microbeads and subsequent magnetic column separation; Miltenyi); the resultant population was >99% pure for DC content, as determined by flow cytometry using CD11c in combination with CD80, CD86, and CD40. Such Treg-conditioned DC were then used as stimulator cells, with degree of proliferation determined relative to DC conditioned with control T cells (cells generated ex vivo from total CD4 cells) or sham-treated DC. MLR assays using pre-conditioned DC were also performed with anti-PD-L1 (20 μg/ml) or isotype control antibody.

### Laser Scanning Cytometry

On day 5, responder T cells were evaluated for expression of PD-L1 binding partners PD1 and CD80. The responder T cells were blocked with a specific αPD1 (1 μg/1×10⁶ cells) and αCD80 (1 μg/1×10⁶ cells) antibody and then PD-L1 binding was studied by incubation with recombinant PD-L1-Fc fusion molecule (R&D); secondary incubation was performed with FITC-labeled rabbit anti-human IgG, Fc-fragment antibody (The Jackson Laboratory, Bar Harbor, Me.). Stained T cells were delivered to 96 well plates with a plastic #1 cover slip bottom (1×10⁵ cells in 200 μl) and analysed (iCys Laser Scanning Cytometer; Compucyte Corporation; Cambridge, Mass.). Cells were scanned (488 nm laser) and fluorescence was detected (530/30 nm band-pass filter). Scan images and fluorescence data were generated (iGeneration and innovator software; Compucyte). Images were collected at 0.5μ scan resolution.

### Xenogeneic GVHD Model

Human effector CD4⁺Th1/CD8⁺Tc1 (Teff) cells were generated by T cell culture for 6 days by co-stimulation and expansion of T cells in rhIL2 (20 IU), αIL-4 (100 ng/ml), rhIL-12 (20 ng/ml), and rapamycin (1 μM). On day 6 of culture, Teff cells were harvested and injected [i.v. by retro-orbital method, as previously described (Nervi et al. (2007) Exp. Hematol. 35(12): 1823-1838)] into Rag2^{-/-}γc^{-/-} mice conditioned with chlodronate and radiation; Teff cell dose was either 1 or 3×10⁷ cells/recipient (higher dose used for evaluation of post-transplant lethality). Specific cohorts additionally received ex vivo generated Tregs (generated from CD4⁺CD127⁻ cells) or control T cells (generated from total CD4 cells) at a dose of 0.5 or 1.5×10⁶ cells/recipient such that the in vivo ratio of effector Teff cells to Tregs always matched that utilized in the allogeneic MLR (20:1). As indicated, cohorts additionally received pooled allogeneic DC (complete mismatch as compared to Teff and Tregs) utilized in the MLR (DC dose, 0.5 or 1.5×10⁶ cells/recipient) to maintain constant ratios; allogeneic DC were either not conditioned or conditioned with ex vivo generated Tregs or control T cells. For blocking experiments, conditioned DC were incubated with anti-PD-L1 (20 μg/ml) or isotype control antibody prior to adoptive transfer. In some cases, anti-PD-L1 was injected following cell transfer (i.p.; 100 μg/recipient). After adoptive transfer, human engraftment was calculated using flow cytometry data from splenic single cell suspensions (% huCD45⁺=[huCD45⁺ (huCD45⁺+mCD45⁺)]×100%). Surface or intra-cellular flow cytometry was performed at indicated days after adoptive transfer to assess in vivo modulation of responder human CD4 and CD8 T cells and human DC.

### Statistical Analysis

Flow cytometry and cytokine data were analyzed using student's 2-tailed t tests. Comparison values of p<0.05 were considered statistically significant. Survival was determined using Kaplan-Meyer's test. In some cases, statistical analyses were performed using the ANOVA method, with p-values adjusted using Dunnett's method. LPS lethality data was analyzed using log rank test.

### For Examples 12-16:

### Mice

Various immune-deficient murine hosts were utilized, depending upon availability. Female RAG2⁻/-cγ^{-/-} mice were obtained from Taconic (Hudson, N.Y.) and utilized at 8-12δ weeks of age. Experiments were performed according to a protocol approved by the NCI Animal Care and Use Committee. Mice were housed in a sterile facility and received sterile water and pellets. As in previously reported methods (van Rijn et al. Blood 102: 2522-2531 (2003); Mutis et al. Clin. Cancer Res. 12: 5520-5525 (2006)), mice were injected with 0.1 ml chlodronate containing liposomes (Encapsula Nanoscience, Nashville, Tenn.) for macrophage depletion and given low-dose irradiation (350 cGy). Female NOD/SCID mice were obtained from Jackson Laboratory (Bar Harbor, Me.) and conditioned with low dose radiation (450 cGy). NODLtSz-scidIL2Rgamma null mice (NSG) (Shultz et al. J. Immunol. 174: 6477-6489 (2005)) were obtained from Jackson Laboratory; NSG hosts did not undergo conditioning prior to human cell transfer.

Human T Cell Transduction with Lentivirus

Normal donor peripheral blood cells were collected by apheresis on an IRB-approved protocol. Total lymphocytes were isolated by elutriation (Abrahamsen et al. J. Clin. Apher. 6: 48-53 (1991)). Total CD4+ T cells were then enriched by CD4 microbeads according to manufacturer instructions. CD4+ T cells were co-stimulated at a 3 bead:1 T cell ratio in media containing IL-2 (20 IU/ml) and rapamycin (1 μM). At day 3 of culture, cells were washed and replated at a concentration of 2×105 cells/ml in media containing IL-2 and lentiviral supernatants at 20 MOI/ml.

### Flow Cytometry

T cells were washed with PBS supplemented with 0.1% BSA and 0.01% azide, and stained using anti-: CD4 APC (clone: RPA-T4), CD19 PE (clone: H1B19), and PDL1 FITC (clone: M1H1). For assessment of cell death, T cells were stained for surface markers (CD4 FITC, CD19 PE and PDL1 PE-cy7), resuspended in Annexin V buffer, and stained with AV APC and 7AAD according to manufacturer's instructions. For in vivo monitoring of human T cells, splenocytes were stained with CD45 FITC (clone: H130), CD3 Pecy5 (clone: H1T3a), PDL1 Pe-cy7, PD1 APC (clone: MIH4), CD80 PE (clone: L307.4), and CD19 APC-cy7 (clone: SJ25C1). For intra-cellular (IC) flow cytometry, fixation and permeabilization buffer was utilized (eBioscience). IC flow cytometry was performed with combinations of CD45 FITC, CD3 Pecy5, PDL1 Pecy7, Tbet APC (clone: 4B10; eBioscience), Foxp3 PB (clone 249D; eBioscience), and CD19 APC-cy7; other reagents for IC flow cytometry included IL-2 FITC (clone: MQ1-17H12), IFN-γ PE (clone: 45.B3), CD3 Pecy5, PDL1 Pecy7, TNF-α APC (clone:MAB11; eBioscience), Foxp3 PB, CD45 APC-cy7 or CD45 FITC, active Caspase-3 PE, CD3 Pecy5, PDL1 Pecy7, PD1 APC, Foxp3 PB, and CD19 APC-cy7.

siRNA Knockdown of PD1

siRNA oligonucleotides for PD1 (P1, P2, P3, and P4) and AllStar Negative control siRNA were purchased from Qiagen (Valencia, Calif.). Transfection of siRNA was performed according to manufacturer's instructions (Amaxa). Transfected cells were costimulated as previously described and harvested for real time PCR, protein, and functional assays at day 3 post-transfection. All in vivo experiments were performed with P4 siRNA, which had the most efficient PD1 knockdown.

### Xenogeneic GVHD Model

Human effector CD4+Th1 (Teff) cells were generated by T cell culture for 6 days with co-stimulation and expansion in rhIL2 (20 IU/ml), anti-IL-4 (100 ng/ml), rhIFN-α 2b (1×106 IU/ml), and rapamycin (1 μM) (Amarnath et al. Autophagy 6). On day 6 of culture, Teff cells were harvested and injected i.v. by retro-orbital injection (Nervi et al. Exp. Hematol. 35: 1823-1838 (2007)) into immune-deficient murine hosts; Teff cell dose was 1×106 cells/recipient. Specific cohorts additionally received ex vivo generated Tregs (generated from CD4+CD127− cells; dose of 0.5×10⁴ cells/recipient); other cohorts received non-polarized CD4+ T cells transduced with control-LV or PDL1-LV (dose of 0.5×10⁴ or 1×10⁶ cells/recipient, as indicated in figure legend). AZT was administered by i.p. injection (twice per day; dose of 2.5 mg/kg/day). After adoptive transfer, human engraftment was calculated using flow cytometry data from splenic single cell suspensions (% huCD45+=[huCD45+(huCD45++mCD45+)]×100%).

### Example 1

Human codon optimized, human PD-L1 cDNA sequence was purchased from GenScript Inc. Human PD-L1 cDNA was designed to be flanked with 5′ XbaI and 3′ BamHI restriction enzyme sites and was sub-cloned into a pUC57 plasmid by GenScript. In a previously constructed shuttle vector, pGEM-4Z-CD19ΔTMPK-IRES-α-Gal-A (Promega), the α-Gal-A cDNA sequence was excised out and replaced with the codon optimized human PD-L1 cDNA sequence excised from pUC57 (GenScript) using the restriction enzyme sites XbaI and BamHI. Next, the CD19ΔTMPK-IRES-PD-L1 sequence was excised from the pGEM-4Z shuttle vector and sub-cloned into pDY.EG.WS. Self-Inactivating (SIN) lentivirus transfer vector containing the Elongation Factor 1-α (EF1-α) promoter and the Woodchuck hepatitis Post-transcriptional Regulatory Element (WPRE) elements upstream and downstream of the CD19ΔTMPK-IRES-PD-L1 transgene, respectively, via EcoRI and BamHI restriction enzyme sites. Dideoxy Chain Termination DNA Sequencing was performed using the following primers to confirm the final sequence (SEQ ID NO: 7): 1WPRE-Reverse: 5′-CACAAATTTTGTAATCCAGAGGTTGATT-3′ (SEQ ID NO:9), 2MidTMPK-Reverse: 5′-CTAGGGAAAAATTCTCCTTGGCA-3′ (SEQ ID NO:10), 3PD-L1-Reverse: 5′-ACACGGCGAAGATCCGCAT-3′ (SEQ ID NO:11) and 4MidCD19-Reverse: 5′-TCCTGGCTGAGGCTCTGGTT-3′ (SEQ ID NO:12).

This example demonstrated a method of making a nucleic acid comprising a nucleotide sequence encoding a TMPK variant, truncated CD19, and PD-L1.

### Example 2

This example demonstrates a method of making a host cell comprising a nucleotide sequence encoding a TMPK variant, truncated CD19, and PD-L1.

Primary human CD4+ T cells were isolated and expanded ex vivo using co-stimulation, type I cytokine polarization, and rapamycin to induce an anti-apoptotic phenotype. Normal donor peripheral blood cells were collected by apheresis on an IRB-approved protocol. Lymphocytes and monocytes were isolated by elutriation (Elutra®; Gambro Systems; Lakewood, Colo.). Lymphocytes were activated by anti-CD3, anti-CD28 co-stimulation (bead:cell ratio, 3:1) and cultured in X-VIVO 20 with 5% heat-inactivated AB serum containing rapamycin (1 μM), rhIL-2 (20 IU/ml), and rhIFN-α2b (10,000 IU/ml). All reagents were only added at day 0, except for IL-2, which was also added on days 2 and 4. Cultures were initiated at 1.5×10⁶ cells/ml and harvested on day 6 for further in vitro or in vivo studies. Culture supernatant containing the CD19/TMPK and PD-L1 transgenes on the lentiviral backbone as prepared in Example 1 was added at day 1 of culture (addition of viral supernatant at 10% of total culture volume).

Four-color flow cytometry was performed to identify human T cells that expressed CD3, CD19, and PD-L1. All antibodies were purchased from Becton-Dickinson. Flow cytometry data was combined with total spleen cell and total bone marrow cell data to quantify the absolute number of transgene-expressing human T cells per spleen or marrow.

At day 6 of culture, ˜1% of transduced cells co-expressed both CD19 and PD-L1, and ˜0% of nontransduced (NT) (control group) cells co-expressed both CD19 and PD-L1. By day 9 of culture, after removal of rapamycin from culture, co-expression of transduced cells increased to ˜3.5% (co-expression of NT cells was less than ˜1%). Finally, at day 12 of culture, after flow cytometry sorting, the frequency of T cells co-expressing the genes had increased to ˜40% (co-expression of NT cells was ˜0%).

This example demonstrated that T cells transduced with a nucleotide sequence encoding a TMPK variant, truncated CD19, and PD-L1 co-express CD19 and PD-L1.

### Example 3

This example demonstrates the adoptive transfer of T cells comprising a nucleotide sequence encoding a TMPK variant, truncated CD19, and PD-L1 into a host.

Female RAG2^{-/-}gc^{-/-} mice were obtained from Taconic (Hudson, N.Y.) and utilized at 8-12 weeks of age. Experiments were performed according to a protocol approved by the NCI Animal Care and Use Committee. Mice were housed in a sterile facility and received sterile water and pellets. Mice were injected with 0.1 ml chlodronate containing liposomes (Encapsula Nanoscience, Nashville, Tenn.) for macrophage depletion and given low-dose irradiation (350 cGy) prior to infusion of 10,000 human T cells (transduced as described in Example 2 or nontransduced) administered by intra-venous injection.

At day 12 of culture, 10,000 transduced T cells (prepared as described in Example 2) or 10,000 nontransduced (NT) T cells (control group) were injected into immune-deficient murine hosts (RAG2^{-/-}cγ^{-/-} mice). On day 5 after adoptive transfer, spleen and bone marrow were collected and subjected to flow cytometry as described in Example 2 to detect human T cells that co-expressed CD19 and PD-L1. Absolute number of cells per organ per mouse (n=5 per cohort) were counted.

In the spleens of mice that received transduced T cells, ˜400×10²T cells that co-expressed CD19 and PD-L1 were detected. With respect to the spleens of mice that received nontransduced cells, ˜0 T cells co-expressing CD19 and PD-L1 were detected.

In the bone marrows of mice that received transduced T cells, ˜7500×10² T cells that co-expressed CD19 and PD-L1 were detected. With respect to the bone marrows of mice that received nontransduced cells, ˜0 T cells co-expressing CD19 and PD-L1 were detected.

This example demonstrated that T cells comprising a nucleotide sequence encoding a TMPK variant, truncated CD19, and PD-L1 co-express CD19 and PD-L1 five days after adoptive transfer.

### Example 4

This example demonstrates the ex vivo expansion of adoptively transferred T cells comprising a nucleotide sequence encoding a TMPK variant, truncated CD19, and PD-L1.

A portion of the cells isolated at day 5 after adoptive transfer in Example 3 were isolated and subjected to repeat co-stimulation and ex vivo expansion for an additional five days. The cells were subjected to flow cytometry as described in Example 2 to detect human T cells that co-expressed CD19 and PD-L1.

Co-expression of CD19 and PD-L1 was detected in ˜300×10² T cells derived from the spleens of mice that received transduced T cells. With respect to the cells derived from the spleens of mice that received nontransduced T cells, ˜0 T cells co-expressing CD19 and PD-L1 were detected.

Co-expression of CD19 and PD-L1 was detected in ˜200×10²T cells derived from the bone marrows of mice that received transduced T cells. With respect to the cells derived from the bone marrows of mice that received nontransduced T cells, ˜0 T cells co-expressing CD19 and PD-L1 were detected.

This example demonstrated that transduced human T cells that underwent an additional five days of ex vivo expansion five days after adoptive transfer continued to co-express CD19 and PD-L1.

### Example 5

This example demonstrates the adoptive transfer of T cells comprising a nucleotide sequence encoding a TMPK variant, truncated CD19, and PD-L1 into a host.

Transduced T cells (prepared as described in Example 2) or nontransduced (NT) T cells (control group) were adoptively transferred into mice as described in Example 3. On day 21 after adoptive transfer, spleen and bone marrow were collected and subjected to flow cytometry as described in Example 2 to detect human T cells that co-expressed CD19 and PD-L1. Absolute number of cells per organ per mouse (n=5 per cohort) were counted.

In the spleens of mice that received transduced T cells, ˜30×10²T cells that co-expressed CD19 and PD-L1 were detected. With respect to the spleens of mice that received nontransduced cells, ˜0 T cells co-expressing CD19 and PD-L1 were detected.

In the bone marrows of mice that received transduced T cells, ˜750×10²T cells that co-expressed CD19 and PD-L1 were detected. With respect to the bone marrows of mice that received nontransduced cells, ˜0 T cells co-expressing CD19 and PD-L1 were detected.

This example demonstrated that T cells comprising a nucleotide sequence encoding a TMPK variant, truncated CD19, and PD-L1 continue to co-express CD19 and PD-L1 twenty-one days after adoptive transfer.

### Example 6

This example demonstrates the ex vivo expansion of adoptively transferred T cells comprising a nucleotide sequence encoding a TMPK variant, truncated CD19, and PD-L1.

A portion of the cells isolated at day 21 after adoptive transfer in Example 5 were isolated and subjected to repeat co-stimulation and ex vivo expansion for an additional six days. The cells were subjected to flow cytometry as described in Example 2 to detect human T cells that co-expressed CD19 and PD-L1.

Co-expression of CD19 and PD-L1 was detected in ˜700×10² T cells derived from the spleens of mice that received transduced T cells. With respect to the cells derived from the spleens of mice that received nontransduced T cells, ˜0 T cells co-expressing CD19 and PD-L1 were detected.

Co-expression of CD19 and PD-L1 was detected in ˜2250×10² T cells derived from the bone marrows of mice that received transduced T cells. With respect to the cells derived from the bone marrows of mice that received nontransduced T cells, ˜0 T cells co-expressing CD19 and PD-L1 were detected.

This example demonstrated that human T cells transduced with a nucleotide sequence encoding a TMPK variant, truncated CD19, and PD-L1 that underwent an additional six days of ex vivo expansion 21 days after adoptive transfer continued to co-express CD19 and PD-L1.

### Example 7

This example demonstrates that Treg cells modulate the programmed death-1 (PD-1) pathway.

Experiments were performed to characterize the mechanism of immune modulation mediated by expanded Tregs generated from CD4⁺CD127⁻ cells. Blockade of TGF-β, IL-10, IDO, CTLA4, or LAP did not abrogate Treg suppression in the allo-MLR. However, experiments utilizing transwell plates indicated that Treg suppression in the allo-MLR was contact dependent. Expression of PD-L1 on bulk CD4 and Treg cells was measured by flow cytometry. Tregs expressed increased PD-L1 (˜15% Tregs expressed CD4+PD-L1+; ˜5% bulk CD4 cells expressed CD4+PD-L1+; mean±SEM of n=4 experiments, p=0.05)

Control CD4 cells and Tregs were co-cultured with pooled allogeneic DC for 24 hrs (Treg cell to DC ratio, 1:1) to generate “DC_{CD4}” and “DC_{Treg}” populations, respectively. DC were also cultured alone (DCₐₗₒₙₑ). Control CD4 cells and Tregs were then removed and resultant conditioned DC were evaluated for co-expression of CD80 and PD-L1. Allogeneic DC isolated from the Treg-containing MLR expressed increased PD-L1 relative to DC isolated from the standard MLR; remarkably, DC harvested from control CD4-containing MLR failed to upregulate PD-L1 (˜4% DC cultured alone expressed CD80+PD-L1+; ˜3% DC_{CD4} expressed CD80+PD-L1+; ˜30% DC_{Treg} expressed CD80+PD-L1+). Of note, Treg-conditioned DC did not have increased expression of PD-1 (CD11c⁺PD-1⁺ cells, <1%; mean±SEM of n=5 experiments; p=0.007).

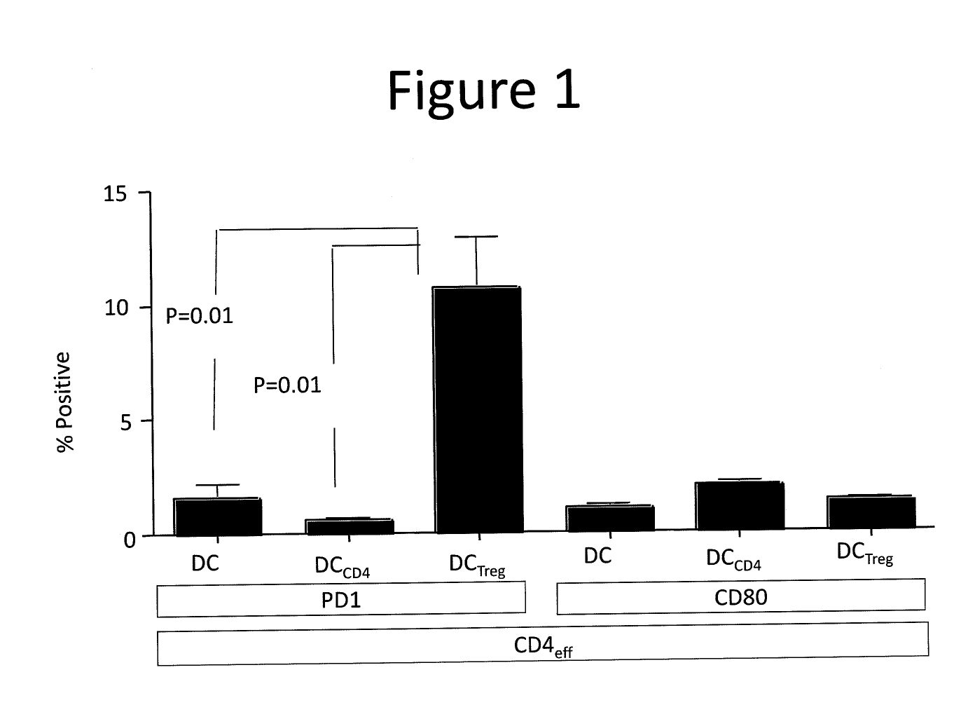

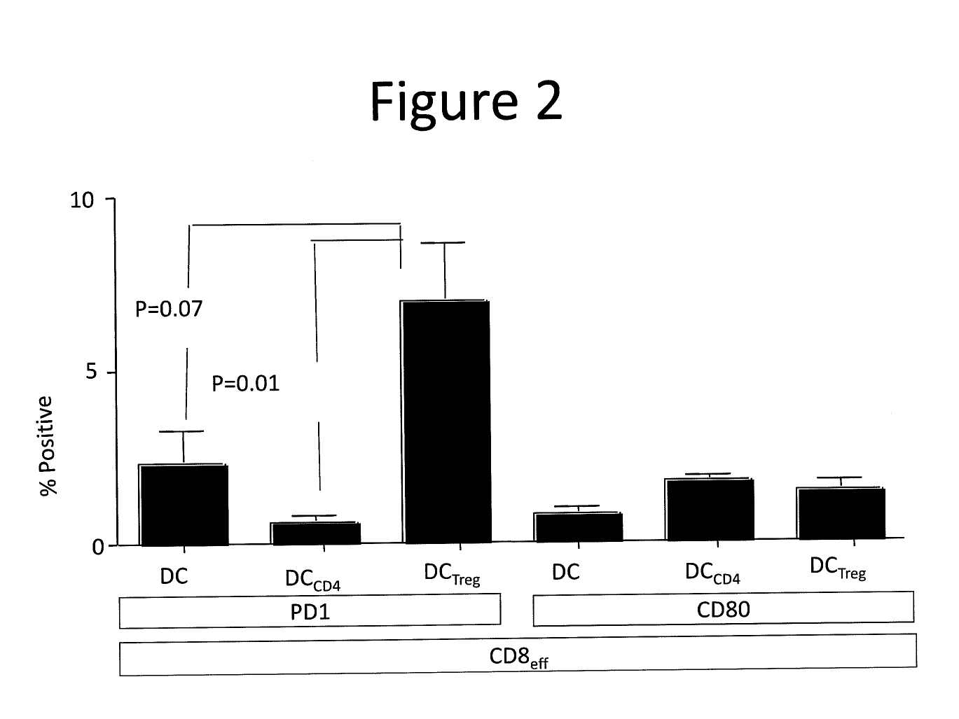

PD-L1 inhibits T cell function via the PD-1 receptor and B7-1 (CD80) (Butte M J et al. (2007) Immunity 27(1): 111-122). To determine PD-L1 binding pathways, effector T cell expression of PD-1 and CD80 was measured after incubation with three types of allogeneic myeloid DC (control, Treg-conditioned, or control CD4-conditioned). PD-L1 binding partners PD1 and CD80 were detected by flow cytometry on effector CD4⁺ cells and CD8⁺ cells after exposure to control or conditioned allogeneic DC for 48 hrs. Summation of isotype control and PD1 staining of CD4 and CD8 effectors (mean±SEM of n=3 experiments) was calculated and results are shown in FIGS. 1 and 2. Effector CD4⁺ T cells (FIG. 1) and CD8⁺ T cells (FIG. 2) upregulated PD-1 expression, but not CD80 expression, upon exposure to Treg-conditioned DC but not CD4-conditioned DC.

A PD-L1 fusion protein was utilized to characterize binding pathways. Laser scanning cytometry (LSC) was used for detection of PD-L1 binding partners on both CD4+ and CD8+ responder T cells. Enriched responders were incubated with a PD-L1 fusion protein. It was found that effector T cells upregulated total PD-L1 binding partners in the presence of Treg-conditioned DC but not CD4-conditioned DC. Blocking studies were performed to determine PD-L1 receptor usage: enriched responders were blocked with anti-PD1 or anti-CD80 and then incubated with PD-L1 fusion protein. Effector T cell PD-L1 binding was abrogated by T cell pre-incubation with anti-PD1 but not anti-CD80: The percentage of cells using PDL1 binding partners was ˜22% for Treg-conditioned DC without blocking antibody and ˜7% for Treg-conditioned DC blocked with anti-PD1 (p=0.05). The percentage of cells using PDL1 binding partners was ˜32% for Treg-conditioned DC blocked with anti-CD80.

Effector T cell PD-L1 binding was quantified by flow cytometry. Allo-MLR was established using control CD4- or Treg-conditioned DC. After 48 hrs, responder T cells were harvested, stained with PD-L1 fusion protein, and flow cytometry was performed. Remarkably, PD-L1 binding was greatly increased on effector T cells exposed to Treg-conditioned DC (% effector T cell PD-L1 binding, increased from 7.3±0.4 to 92.6±2.8, p=0.001; mean±SEM n=3 donors). Blocking studies were performed. Similar to results using LSC, effector T cell PD-L1 binding was abrogated by T cell pre-incubation with anti-PD1 but not anti-CD80. The percentage of cells using PDL1 binding partners was ˜28% for Treg-conditioned DC without blocking antibody and ˜5% for Treg-conditioned DC with anti-PD1 antibody (p=0.03; mean±SEM). The percentage of cells using PDL1 binding partners was ˜32% for Treg-conditioned DC blocked with anti-CD80.

This example demonstrated that Treg-conditioned DC affect PD-L1 binding of effector T cells.

### Example 8

This example demonstrates that Treg-conditioned DC have reduced allostimulatory function in part through PD-L1.

Secondary transfer experiments were performed to evaluate whether Tregs mediated suppression in part through DC modulation. Control CD4 cells or Tregs were generated ex vivo, and then utilized to condition allogeneic DC (24 hr incubation; 1:1 cell ratio). Conditioned DC were then purified by negative selection using anti-CD3 microbeads and utilized as the stimulator population (DC to responder T cell ratio, 1:20). The allo-MLR was performed in the presence of anti-PD-L1 or isotype control antibody. CFSE dye dilution proliferation assays were performed to determine responder CD4 alloreactivity in response to: unmodified DC; DC conditioned with Tregs either without or with addition of anti-PD-L1; and DC conditioned with control CD4 cells either without or with anti-PD-L1. Representative result for CFSE dye dilution proliferation assay to determine responder CD4 alloreactivity in response to unmodified DC was 83%.

Percent inhibition of responder CD4 cell proliferation and responder CD8 cell proliferation were calculated relative to proliferation measured using sham-treated DC. The pooled results are shown in Table 1 (mean±SEM of n=8 normal donors). Allogeneic DC conditioned with Tregs yielded reduced levels of CD4⁺ and CD8⁺ responder T cell proliferation relative to CD4-conditioned allogeneic DC (Table 1). Blockade of DC expression of PD-L1 partially corrected the observed stimulatory deficit of Treg-conditioned DC on CD4⁺ and CD8⁺ T cell proliferation (Table 1).

TABLE 1

| | Without anti- | With anti- | Without anti- | With anti- |
|---|---|---|---|---|
| | PDL1 | PDL1 | PDL1 | PDL1 |
| | DC_{CD4} | | DC_{Treg} | |
| % CD4 | ~5 | 0 | ~40 | ~10 |
| Suppression | | | p = 0.04 | |
| % CD8 | 0 | 0 | ~50 | ~20 |

| | |
|---|---|
| Suppression | p = 0.031 |

This example demonstrated that Treg-conditioned DC reduces proliferation of CD4⁺ and CD8⁺ responder T cells, and that blocking DC expression of PD-L1 partially reverses this reduction of T cell proliferation.

### Example 9

This example demonstrates that Treg-conditioned DC modulate effector T cells in vivo via PD-L1.

An in vivo xenogeneic transplantation model was used to further characterize the ability of Tregs or Treg-conditioned DC to modulate the PD1 pathway. The xenogeneic transplantation model utilized Rag2^{-/-}γc^{-/-} mice that received some combination of human cells, including: CFSE-labeled effector Teff cells (“Teff”); untreated DC (“DC”), control CD4-conditioned DC (“DC_{CD4}”), or Treg-conditioned DC (“DC_{Treg}”); and ex vivo generated control CD4 cells (“CD4”) or regulatory T cells (“Treg”). Spleens were harvested 24 hrs after cell infusion, and analyzed by flow cytometry. Human cells were gated by human CD45⁺ staining, including any human CD3⁺ T cells. PDL1 expression was evaluated on DC by CD11c staining and PD1 expression was evaluated on CD4 cells. Flow cytometric analysis was used to measure the absolute number of: CD11c⁺ DC that co-expressed PD-L1; CD8⁺ and CD4⁺ T cells that co-expressed PD-L1; and CD8⁺ and CD4⁺ T cells that co-expressed PD-1. Results are shown in Table 2 (mean±SEM of n=5 mice per cohort; p values given are with respect to DC_{Treg}).

TABLE 2

DC

| | | | |
|---|---|---|---|
| CD4 | Treg | DC_{CD4} | DC_{Treg} |

Teff

| | | | | | | | | |
|---|---|---|---|---|---|---|---|---|
| Number of | ~12 | (p = .068) | ~11 | (p = .036) | ~10 | (p = .031) | ~17 (p = .073) | ~40 |
| CD11c + PDL1 + | | | | | | | | |
| Cells (10³) | | | | | | | | |
| Number of | ~10 | (p = .039) | ~11 | (p = .037) | ~10 | (p = .033) | ~12 (p = .055) | ~60 |
| CD8 + PDL1 + | | | | | | | | |
| Cells (10³) | | | | | | | | |
| Number of | ~9 | (p = .055) | ~8 | (p = .003) | ~7 | (p = .006) | ~12 (p = .041) | ~27 |
| CD4 + PDL1 + | | | | | | | | |
| Cells (10³) | | | | | | | | |
| Number of | ~7 | (p = .029) | ~6 | (p = .022) | ~7 | (p = .024) | ~8 (p = .041) | ~35 |
| CD8 + PD1 + | | | | | | | | |
| Cells (10³) | | | | | | | | |
| Number of | ~9 | (p = .041) | ~9 | (p = .019) | ~8 | (p = .009) | ~10 (p = .027) | ~25 |
| CD4 + PD1 + | | | | | | | | |
| Cells (10³) | | | | | | | | |

Recipients of Treg-conditioned DC, which expressed increased PD-L1 in vitro prior to adoptive transfer, had an increased in vivo number of dendritic cells in the spleen that expressed PD-L1 (Table 2); relative to recipients of control DC, recipients of Treg-conditioned DC also had an increase in PD-L1 expressing DC in the bone marrow (p=0.006). Remarkably, recipients of Treg-conditioned DC also had increased numbers of effector CD4⁺ and CD8⁺ T cells in the spleen that expressed PD-L1 in vivo (Table 2); such recipients also had increased numbers of T cells that expressed PD-L1 in the bone marrow (p=0.003). In marked contrast, recipients of control CD4-conditioned DC did not have increased responder T cell PD-L1 expression. Interestingly, recipients of Treg-conditioned DC also had increased numbers of effector CD8⁺ and CD4⁺ cells in the spleen that expressed PD-1 in vivo (Table 2); in the bone marrow, such recipients also had increased numbers of CD8⁺PD-1⁺ cells (p=0.02) and CD4⁺PD-1⁺ cells (p=0.009).

Further experiments were performed to assess the functional significance of this sequential increase in PD-L1 expression from Treg cell, to conditioned DC, and then to responder T cells in vivo. Control CD4-conditioned or Treg-conditioned DC were incubated for 30 min. with anti-PD-L1 or isotype control antibody prior to adoptive transfer; in addition, anti-PD-L1 or isotype control antibody was injected i.p. immediately after cell transfer (100 μg/mouse). Spleens were harvested 24 hrs after cell infusion, and analyzed by flow cytometry to determine the absolute number of: CD11c⁺ DC that co-expressed PD-L1); CD8⁺ and CD4⁺ T cells that co-expressed PD-L1; and CD8⁺ and CD4⁺ T cells that co-expressed PD-1. The results are shown in Table 3 (mean±SEM of n=5 mice per cohort; p values are with respect to DC incubated without antibody).

TABLE 3

| | | α-PDL1 | Isotype control Ab |
|---|---|---|---|
| | DCTreg | | |
| | Teff | | |
| Number of | ~405 | ~200 (p = .07) | ~399 |
| CD11c + PDL1 + | | | |
| Cells (10³) | | | |
| Number of | ~550 | ~250 (p = .09) | ~550 |
| CD8 + PDL1 + | | | |
| Cells (10²) | | | |
| Number of | ~7.4 | ~4 | ~5 |
| CD4 + PDL1 + | | | |
| Cells (10³) | | | |
| Number of | ~170 | ~90 (p = .03) | ~180 |
| CD8 + PD1 + | | | |
| Cells (10²) | | | |
| Number of | ~8 | ~5 | ~9 |
| CD4 + PD1 + | | | |
| Cells (10³) | | | |

Further experiments were performed to confirm the specificity of this apparent transfer of PD-L1 status from Treg cell, to conditioned DC, and then to responder T cells in vivo. Recipients of Treg-conditioned DC that were incubated with anti-PD-L1 prior to adoptive transfer tended to have reduced numbers of PD-L1 expressing DC in vivo (Table 3); similarly, a similar trend towards reduced in vivo numbers of PD-L1 expressing DC in the antibody-treated cohort was observed in the bone marrow (p=0.08). However, blockade of PD-L1 on Treg-conditioned DC yielded a reduction in the in vivo number of effector CD8 cells expressing PD-L1 (Table 3); a similar reduction in the number of CD8⁺PDL1⁺ cells was observed in the bone marrow (p=0.01). Blockade of PD-L1 on Treg-conditioned DC also reduced the number of PD-L1 expressing responder effector CD4⁺ cells in the spleen (Table 3) and in the bone marrow (p=0.02). Finally, PD-L1 blockade of Treg-conditioned DC reduced the in vivo number of effector CD8⁺ cells in the spleen that expressed PD-1 (Table 3); the number of CD8⁺PD1⁺ cells in the bone marrow or the number of CD4⁺PD1⁺ T cells in the spleen was not significantly altered by PD-L1 blockade (Table 3).

This example demonstrated that PD-L1 expression on Treg-conditioned DC is functionally significant in vivo, particularly with respect to downstream expression of PD-L1 on effector CD4 cells.

### Example 10

This example demonstrates that Tregs or Treg-conditioned DC protect against lethal xenogeneic GVHD in vivo via PD-L1.

Using Rag2^{-/-}γc^{-/-} mice as host, transplant cohorts received effector T cells (“T_{eff}”) in combination with allogeneic DC, control CD4 cell-conditioned DC (“DC_{CD4}”), or Treg-conditioned DC (“DC_{Treg}”); other cohorts received T_{eff} cells in combination with allogeneic DC plus either Treg cells (“Treg”) or control CD4 cells (“CD4”). The doses of the T_{eff}, DC, and Treg cells was 3×10⁷, 3.0×10⁶, and 1.5×10⁶ cells per recipient, respectively. Xenogeneic GVHD was evaluated by weight loss measurement, survival analysis, and histology evaluation of GVHD target tissues.

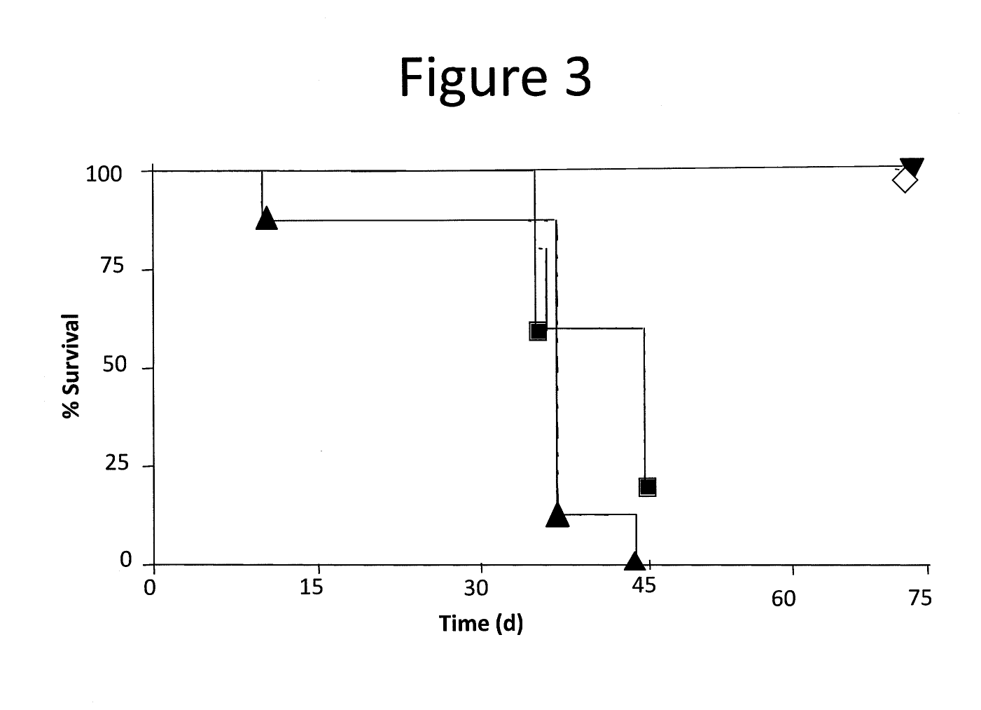

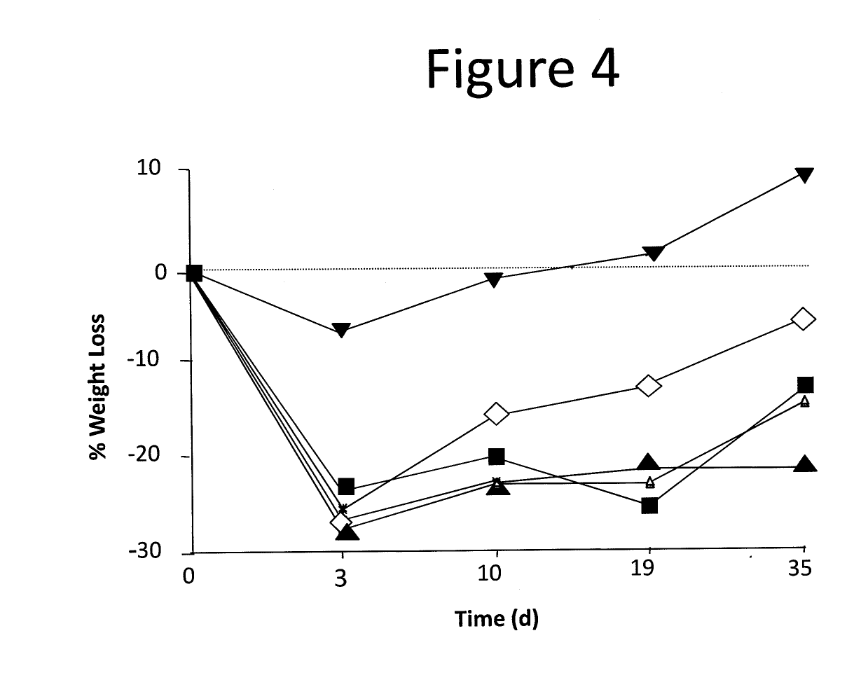

As shown in FIG. 3, recipients of Tregs (▾) or Treg-conditioned DC (white diamond) were uniformly protected against lethal xenogeneic GVHD, while recipients of control CD4-conditioned DC (▴) uniformly died of xenogeneic GVHD. Post-transplant weight loss, which is a more sensitive clinical parameter of xenogeneic GVHD, was moderated by Treg-conditioned DC (white diamond) therapy and virtually eliminated by Treg cell therapy (▾), as shown in FIG. 4.

In a second experiment, the ability of Treg-conditioned DC to completely abrogate the generation of lethal xenogeneic GVHD was confirmed. Transplants were performed at these same cell doses and post-transplant survival was determined. Treg-conditioned DC were incubated with anti-PD-L1 (“aPDL1”) or isotype control antibody (“mIgG2a”) for 30 min. prior to adoptive transfer; in addition, anti-PD-L1 or isotype control antibody was injected i.p. immediately after cell transfer (100 μg/mouse).

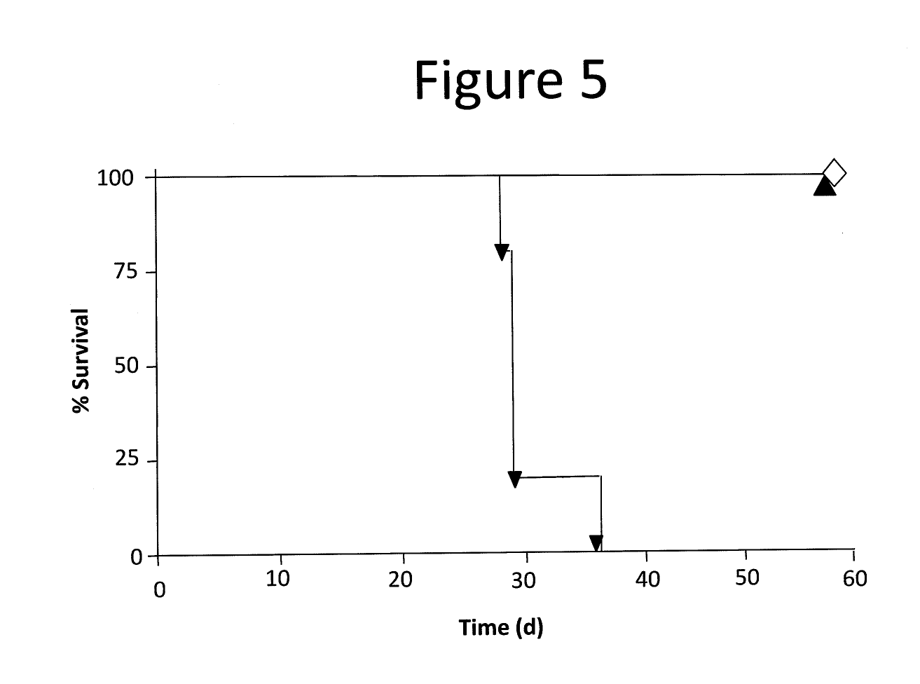

Protection against lethal xenogeneic GVHD conferred by the Treg-conditioned DC was completely abrogated by anti-PD-L1 (▾) but not by isotype control antibody (▴), as shown in FIG. 5. Of note, both control DC and Treg-conditioned DC engrafted and persisted in vivo; such numbers were not substantially influenced by Treg cell therapy or anti-PDL1 antibody. That is, at day 25 post-transplant, the absolute numbers of CD11c⁺ DC per spleen (each value, ×10³; n=5 per cohort) in transplant recipients that received effector human T cells in combination with the indicated specific type of human DC were 136±11 (control DC), 107±6 (control DC and Treg therapy), 418±98 (Treg-conditioned DC), 163±63 (Treg-conditioned DC, anti-PDL1 treated), and 279±77 (Treg-conditioned DC, isotype antibody treated) [each comparison, p=NS by ANOVA test]. GVHD control mice uniformly developed a diffuse skin rash and hair loss; skin histology analysis documented cutaneous acanthosis and hyperkeratosis in GVHD controls but not in Treg cell recipients. Furthermore, GVHD controls, but not Treg cell recipients, developed diffuse lymphocytic infiltration of the liver.

This example demonstrated that Tregs or Treg-conditioned DC protect mice against lethal xenogeneic GVHD, and that this protection is abrogated by anti-PD-L1 antibody.

### Example 11

This example demonstrates that adoptive transfer of host cells expressing a nucleotide sequence encoding a TMPK variant, truncated CD19, and PD-L1 into a murine host prevents xenogeneic GVHD in vivo.

Host mice consisted of immune incompetent Rag2^{-/-}cg^{-/-} mice. Prior to transplantation with human cells, such murine hosts were conditioned with 350 cGy total body irradiation and injected with chlodronate-containing liposomes to further eliminate host cells that might provide resistance to human T cell engraftment. After this host treatment, mice received some combination of human cells, as indicated in Table 4 below. All cells were injected on the same day (“Day 0”) by separate intravenous injection. Such human cells consisted of the following functional types: (1) purified human monocytes (“hMo”; isolated by counterflow centrifugal elutriation; injected at dose of 3×10⁶ cells per host); (2) ex vivo manufactured human CD4⁺ and CD8⁺ T cells that were polarized to a type I cytokine profile and rendered rapamycin-resistant (“Th1/Tc1.R cells”; injected at dose of 1×10⁶ cells per host); (3) ex vivo manufactured human CD4⁺ T cells that were infected with the lentiviral supernatant containing a vector encoding the CD19/TMPK fusion protein but not PD-L1 protein (“Th0-CD19/TMPK”) negative control gene therapy; T cells were manufactured in media that did not contain cytokine polarizing cytokines and did contain rapamycin to render the transgene-expressing T cells rapamycin-resistant; injected at dose of 1×10⁶ cells per host); (4) ex vivo manufactured human CD4⁺ T cells that were infected with the lentiviral supernatant containing a vector encoding the CD19/TMPK fusion protein and also the PD-L1 protein (“Th0-CD19/TMPK/PD-L1”) (SEQ ID NO: 7); positive control gene therapy; T cells were manufactured in media that did not contain cytokine polarizing cytokines and did contain rapamycin to render the transgene-expressing T cells rapamycin-resistant; injected at dose of 1×10⁶ cells per host); and (5) control human CD4+ regulatory T cells (“Tregs”; manufactured ex vivo by CD127 negative selection and subsequent ex vivo expansion in media containing IL-2 and rapamycin; injected at dose of 0.5×10⁶ cells per host). On day 5 after intravenous administration of some combination of the above-listed human cell components, the murine recipients were injected (challenged) by the intravenous injection of bacterial lipopolysaccharide (LPS) to induce a cytokine storm typical of xenogeneic graft-versus-host disease (“x-GVHD”). Mice were then observed for the presence or absence of lethality for a 24 hour time interval. The results are shown in Table 4.

TABLE 4

| | | Human Cell Infusion | | | | | |
|---|---|---|---|---|---|---|---|
| | | | Th1/ | Transgene | | | Sur- |
| | # per | Mono- | Tc1.R | Expressing | Treg | | vival |
| Cohort | Cohort | cytes | Cells | T Cells | Cells | LPS | (%) |
| 1 | 10 | YES | NO | NO | NO | YES | 100 |
| 2 | 10 | YES | YES | NO | NO | YES | 0 |
| 3 | 10 | YES | YES | NO | YES | YES | 80 |
| 4 | 10 | YES | YES | YES | NO | YES | 20 |
| | | | | (Th0- | | | |
| | | | | CD19/ | | | |
| | | | | TMPK) | | | |
| 5 | 10 | YES | YES | YES | NO | YES | 100 |
| | | | | (Th0- | | | |
| | | | | CD19/ | | | |
| | | | | TMPK/ | | | |
| | | | | PD-L1) | | | |

As shown in Table 4, injection of human Th1/Tc1.R cells into the immune-deficient murine hosts uniformly resulted in lethal human-into-mouse xenogeneic GVHD that was induced by LPS challenge (cohort #2, lethality in 10/10 recipients). In marked contrast, injection of this otherwise lethal human Th1/Tc1.R cell inocula and further human T cells that were forced to express CD19/TMPK/PD-L1 (SEQ ID NO: 7) resulted in survival in 100% of recipients (cohort #5). A control cohort that received the further administration of transgene-modified human T cells that did not express PD-L1 were not uniformly protected (cohort #4). A further control cohort that received the further administration of human regulatory T cells was protected against x-GVHD in 80% of recipients (cohort #3).

This example demonstrated that adoptive transfer of T cells expressing SEQ ID NO: 7 into mice effectively protected the mice against xenogeneic GVHD.

### Example 12

This example demonstrates that adoptive transfer of host cells expressing a nucleotide sequence encoding a TMPK variant, truncated CD19, and PD-L1 into a murine host prevents xenogeneic GVHD in vivo.

T cells transduced with CD19/TMPK/PD-L1 (SEQ ID NO: 7; PDL1-LV) were next compared with T_{REG} cells for their capacity to prevent xenogeneic graft-versus-host disease (xGVHD). Th1-polarized human T cells (T_{EFF}) that primarily expressed T-bet (Francisco et al. J. Exp. Med. 206: 3015-29 (2009); Tang et al. J. Exp. Med. 199: 1455-65 (2004)) were adoptively transferred alone (cohort 1) or with either: control-LV-transduced T cells (T_{TMPK}), which express TMPK.CD19 fusion protein without PDL1 (cohort 2); T_{REGS} (cohort 3); or with PDL1-LV-transduced T cells (T_{PDL1}) (cohort 4). At day 5 after adoptive transfer, absolute number of human T cells producing IFN-γ was quantified by flow cytometry. Serum was harvested 90 min later for measurement of TNF-α. The results are shown in Table 5A.

TABLE 5A

| | No. T cells expressing | |
|---|---|---|
| | IFN-γ (×10²) | TNF-α (pg/ml) |
| T_{EFF} | ~22 | ~39 |
| T_{EFF} & T_{TMPK} | ~18 | ~37 |
| T_{EFF} & T_{REG} | ~9 | ~20 |
| T_{EFF} & T_{PDL1} | ~8 | ~21 |

Hosts were subsequently challenged with LPS to induce cytokine-mediated xGVHD (Latchman et al. Nat. Immunol. 2: 261-68 (2001)). The results are shown in Table 5B.

TABLE 5B

| Cohorts | Human T | Human | Regulatory | Post LPS |
|---|---|---|---|---|
| (n) | cells | Monos | cells | Survival (%) |
| 1 (12) | ✓ | ✓ | — | 0 |
| 2 (12) | ✓ | ✓ | T_{TMPK} | 15 |
| 3 (12) | ✓ | ✓ | T_{REG} | 75 |
| 4 (15) | ✓ | ✓ | T_{PDL1} | 80 |

As shown in Table 5, T_{REG} cells and PDL1-LV-transduced T cells similarly inhibited T cell IFN-γ production, systemic TNF-α production, and lethality. Long-term protection of mice from xGVHD was noted 65 days post-transplant in the T_{PDL1} or T_{REG} cohorts.

### Example 13

This example demonstrates that PDL1-LV transduced T cells shift Th1 effector cells towards a regulatory phenotype.

To confirm that therapy using PDL1-LV transduced T cells operated by Th1 cell tolerance induction rather than clonal deletion, the fate of CFSE-labeled T_{EFF} cells was monitored in vivo. PDL1-LV-transduced T cells did not impair T_{EFF} cell engraftment, thereby ruling out a clonal deletion mechanism.

Cohorts of mice received T_{EFF} alone or with either T_{TMPK} or PDL1-LV-transduced T cells (T_{PDL1}), followed by LPS administration on day 5 post-transfer for assessment of lethal cytokine-mediated xGVHD. PDL1-LV-transduced T cells had a modest capacity to inhibit in vivo T_{EFF} cell production of IFN-γ. The absolute splenic numbers for human Th1 cells expressing IFN-γ were ˜1.6×10³ (T_{EFF}); ˜1.3×10³ (T_{TMPK}) and ˜1.0×10³ (T_{PDL1}).

In a reciprocal manner, PDL1-LV transduced T cells increased the absolute number of T_{EFF} cells that expressed Foxp3 in the absence of IFN-γ secretion (˜0.5×10³ (T_{EFF}), ˜0.3×10³ (T_{TMPK}), ˜1.0×10³ (T_{PDL1})). No significant changes were observed with respect to the ability of PDL1-LV transduced T cells to modulate T_{EFF} cell IL-2 or TNF-α secretion without or with Foxp3 expression.

The number of Th1 cells expressing T-bet was also determined. PDL1-modulated T_{EFF} cells had reduced expression of T-bet in vivo (˜100×10³ (T_{EFF}), ˜75×10³ (T_{TMPK}), ˜60×10³ (T_{PDL1})). As such, therapy using PDL1-LV transduced T cells initiated a conversion of Th1 cells towards a regulatory phenotype.

This example demonstrated that therapy using PDL1-LV transduced T cells inhibits in vivo T_{EFF} cell production of IFN-γ, increases the number of T_{EFF} cells that express Foxp3 in the absence of IFN-γ secretion, and initiates a conversion of Th1 cells towards a regulatory phenotype.

### Example 14

This example demonstrates that therapy using PDL1-LV transduced T cells converts human T_{EFF} to a tolerogenic phenotype via a PD1 receptor-specific mechanism.

To identify the specificity of the therapy using PDL1-LV transduced T cells, in vivo experiments were performed using T_{EFF} cells deficient in PD1 receptor via an siRNA approach (T_{EFF}PD1^{kd}). NSG host mice received a combination of human Th1 cells (either unmodified, control siRNA-treated, or PD1 siRNA-treated) and PDL1-LV transduced T cells. At day 6, the absolute number of human T cells and PD1-expressing T cells were enumerated in the spleen. T_{EFF}PD1 ^{kd} cells engrafted similar to control T_{EFF} cells. Consistent with the previous finding that PDL1 caused a trans-up-regulation of PD14, control T_{EFF} cells up-regulated PD1 during therapy using PDL1-LV transduced T cells; however, engrafted T_{EFF}PD1^{kd} cells did not up-regulate PD1.

Splenocyes were stimulated (PMA/ionomycin) and the absolute number of Foxp3-negative T cells expressing IFN-γ, IL-2, and TNF-α were quantified; conversely, the number of Foxp3-positive T cells deficient in production of IFN-γ, IL-2, and TNF-α were quantified. The results are shown in Table 6.

TABLE 6

| | | | T_{EFF}, | T_{EFF}, |
|---|---|---|---|---|
| | | T_{EFF} & | PDL1-LV | PDL1-LV |
| | | PDL1-LV | transduced | transduced |
| | | transduced | T cells, and | T cells, and |
| | T_{EFF} | T cells | control siRNA | PD1 siRNA |
| Absolute no. of | ~0.8 | ~0.4 | ~0.25 | ~0.6 |
| Foxp3− T cells | | | | |
| expressing IFN-γ | | | | |
| (×10³) | | | | |
| Absolute no. of | ~1.75 | ~1 | ~2 | ~2.5 |
| Foxp3− T cells | | | | |
| expressing IL-2 | | | | |
| (×10³) | | | | |
| Absolute no. of | ~3.1 | ~1.5 | ~0.6 | ~2.0 |
| Foxp3− T cells | | | | |
| expressing TNF-α | | | | |
| (×10³) | | | | |
| Absolute no. of | ~0.4 | ~1.0 | ~1.5 | ~0.25 |
| Foxp3 + T cells | | | | |
| expressing IFN-γ | | | | |
| (×10³) | | | | |
| Absolute no. of | ~0.3 | ~1.25 | ~1.6 | ~0.4 |
| Foxp3 + T cells | | | | |
| expressing IL-2 | | | | |
| (×10³) | | | | |
| Absolute no. of | ~0.4 | ~1.3 | ~1.6 | ~0.5 |
| Foxp3 + T cells | | | | |
| expressing TNF-α | | | | |
| (×10³) | | | | |

Therapy using PDL1-LV transduced T cells reduced T_{EFF} cell in vivo effector function, as indicated by reduced numbers of Foxp3-negative IFN-γ positive, IL-2 positive, and TNF-α-positive cells (Table 6); conversely, engrafted T_{EFF}PD1^{kd} cells were relatively resistant to the down-regulatory effect of therapy using PDL1-LV transduced T cells. Furthermore, whereas therapy using PDL1-LV transduced T cells increased the number of T_{EFF} cells that expressed Foxp3 in the absence of effector cytokine secretion (Table 6), T_{EFF}PD1 ^{kd} cells did not express this regulatory phenotype in vivo.

During PDL1 therapy using PDL1-LV transduced T cells, T_{EFF}PD1^{kd} cells were fully capable of stimulating a systemic TNF-α response, maintained expression of T-bet, and were enriched in their capacity to mediate lethal xGVHD (Tables 7A and 7B).

TABLE 7A

| | Tbet+ (×10³) | TNF-α (μg/ml) |
|---|---|---|
| T_{EFF} | ~12 | ~0.2 |
| T_{EFF} & T_{PDL1} | ~4 | ~0.03 |
| T_{EFF} & T_{PDL1} with A647 Neg. siRNA | ~5 | ~0.01 |
| T_{EFF} & T_{PDL1} with PD1 siRNA | ~10 | ~0.2 |

TABLE 7B

| | | | Other | | |
|---|---|---|---|---|---|
| | Human | | treatment of | | Post LPS |
| Cohorts | T_{EFF} | Human | human Teff | Regulatory | survival |
| (n) | cells | Monos | (125 pmol) | cells | (%) |
| 1 (10) | ✓ | ✓ | None | None | 0 |
| 2 (10) | ✓ | ✓ | None | T_{PDL1} | 80 |
| 3 (10) | ✓ | ✓ | A647 | T_{PDL1} | 100 |
| | | | Neg. siRNA | | |
| 4 (11) | ✓ | ✓ | PD1 siRNA | T_{PDL1} | 27 |

This example demonstrated that PDL1-mediated a conversion of human T_{EFF} cells from a pathogenic Th1 phenotype to a tolerogenic T_{REG} phenotype through a mechanism involving PD1 receptor.

### Example 15

This example demonstrates that the TMPK cell fate safety switch is functional in vitro and in vivo.

An ability to precisely titrate the extent and kinetics of CD19/TMPK/PD-L1 gene delivery would facilitate the fine tuning of tolerance induction. Therefore the TMPK cell fate axis that was incorporated into the PDL1 vector was tested. Human CD4+ T cells were transduced with PDL1-LV or a control eGFP-LV and challenged in vitro with AZT prodrug.

In vitro, eGFP-transduced T cells were minimally sensitive to AZT-mediated death. In contrast, PDL1-LV-transduced T cells underwent AZT-mediated death within 72 hours; adoptive transfer of post-AZT viable cells did not result in significant human cell engraftment, thereby indicating that T cell resistance to AZT was minimal. Finally, in vivo AZT therapy resulted in marked and specific in vivo depletion of human T cells expressing CD19/TMPK fusion protein.

### Example 16

This example demonstrates that treating T cells with pervanadate or NSC87877 renders them resistant to the tolerizing effect of PDL1.

Because PD1 receptor signaling occurs via SHP1/2, it was next hypothesized that ex vivo pharmacologic inhibition of this pathway using prevanadate and NSC87877 might also generate T_{EFF} cells (T_{EFF}Shp1/2ⁱⁿ) resistant to the tolerizing effect of PDL1. NSG host mice received a combination of human Th1 cells (either unmodified, pervanadate-treated, or NSC87877-treated) and PDL1-LV transduced T cells. At day 6, absolute numbers of human T cells engrafted in the spleen were enumerated. Splenocyes were stimulated (PMA/ionomycin) and the absolute number of Foxp3-negative T cells expressing IFN-γ (i), IL-2 (ii), and TNF-α (iii) were quantified; conversely, the number of Foxp3-positive T cells deficient in production of IFN-γ (iv), IL-2 (v), and TNF-α (vi) were quantified. The results are shown in Table 8.

TABLE 8

| | | unmodified | pervanadate- | NSC87877- |
|---|---|---|---|---|
| | | T_{EFF} & | treated T_{EFF} | treated T_{EFF} |
| | unmod- | PDL1-LV | & PDL1-LV | & PDL1-LV |
| | ified | transduced | transduced | transduced |
| | T_{EFF} | T cells | T cells | T cells |
| Absolute no. of | ~0.8 | ~0.4 | ~1.5 | ~1.1 |
| Foxp3− T cells | | | | |
| expressing IFN-γ | | | | |
| (×10³) | | | | |
| Absolute no. of | ~1.7 | ~1.0 | ~2 | ~2.6 |
| Foxp3− T cells | | | | |
| expressing IL-2 | | | | |
| (×10³) | | | | |
| Absolute no. of | ~3.0 | ~1.5 | ~3.9 | ~6.0 |
| Foxp3− T cells | | | | |
| expressing TNF-α | | | | |
| (×10³) | | | | |
| Absolute no. of | ~0.4 | ~1.0 | ~.3 | ~0.1 |
| Foxp3+ T cells | | | | |
| expressing IFN-γ | | | | |
| (×10³) | | | | |
| Absolute no. of | ~0.4 | ~1.2 | ~1.3 | ~1.4 |
| Foxp3+ T cells | | | | |
| expressing IL-2 | | | | |
| (×10³) | | | | |
| Absolute no. of | ~0.4 | ~1.1 | ~0.4 | ~1.3 |
| Foxp3+ T cells | | | | |
| expressing TNF-α | | | | |
| (×10³) | | | | |

Although SHP1/2 inhibition did not impair T_{EFF} cell engraftment, T_{EFF} SHP1/2ⁱⁿ cells maintained their effector phenotype in vivo in the presence of PDL1-LV-transduced T cells, as indicated by preserved or increased expression of IFN-γ, IL-2, and TNF-α in the absence of Foxp3 and generally reduced in vivo expression of these effector cytokines in the presence of Foxp3 (Table 8).

Recipients were challenged with LPS and serum was harvested 120 min later for measurement of TNF-α. Absolute numbers of engrafted human T cells expressing T-bet were quantified. The results are shown in Table 9.

TABLE 9

| | | unmodified | pervanadate- | NSC87877- |
|---|---|---|---|---|
| | | T_{EFF} & | treated T_{EFF} | treated T_{EFF} |
| | unmod- | PDL1-LV | & PDL1-LV | & PDL1-LV |
| | ified | transduced | transduced | transduced |
| | T_{EFF} | T cells | T cells | T cells |
| Tbet+ (×10³) | ~1.6 | ~1.0 | ~2.0 | ~2.7 |
| TNF-α (μg/ml) | ~0.2 | ~0.05 | ~0.37 | ~0.42 |

Cohorts of host mice received the human cell inocula specified, followed by LPS administration on day 5 post-transfer for assessment of lethal cytokine-mediated xGVHD. The results are shown in Table 10.

TABLE 10

| | | | Other | | |
|---|---|---|---|---|---|
| | Human | | treatment of | | Post LPS |
| Cohorts | T_{EFF} | Human | human Teff | Regulatory | survival |
| (n) | cells | Monos | (125 pmol) | cells | (%) |
| 1 (10) | ✓ | ✓ | None | | 0 |
| 2 (10) | ✓ | ✓ | None | T_{PDL1} | 80 |
| 3 (12) | ✓ | ✓ | Pervanadate | T_{PDL1} | 33 |
| | | | (100 μM) | | |
| 4 (10) | ✓ | ✓ | NSC87877 | T_{PDL1} | 0 |
| | | | (100 μM) | | |

Finally, in the presence of PDL1-LV-transduced T cells, T_{EFF}SHP1/2ⁱⁿ cells were enhanced in their ability to promote systemic TNF-α production, had preserved or increased expression of T-bet, and were potent mediators of lethal xGVHD (Tables 9 and 10).

This example demonstrated that blockade of the PD1 receptor or down-stream SHP1/2 signaling using pervanadate or NSC87877 prevents PDL1-mediated suppression of human effector T cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nucleic acid comprising a nucleotide sequence encoding programmed death ligand-1 (PD-L1) and a nucleotide sequence encoding a fusion protein comprising thymidylate kinase (TMPK) or a variant thereof and a cell surface marker or a variant thereof.

2. The nucleic acid according to claim 1, wherein the cell surface marker or variant thereof is CD19, truncated CD19, CD25, low affinity nerve growth factor receptor (LNGFR), truncated LNGFR, CD34, truncated CD34, erythropoietin receptor (EpoR), HSA or CD20.

3. The nucleic acid according to claim 1, wherein the cell surface marker or variant thereof is CD19 or truncated CD19.

4. The nucleic acid according to claim 3, wherein the nucleotide sequence encoding a fusion protein comprising CD19 or truncated CD19 comprises SEQ ID NO: 30, 33, or 38.

5. The nucleic acid according to claim 1, wherein the nucleotide sequence encoding a fusion protein comprising TMPK or a variant thereof comprises a nucleotide sequence encoding TMPK selected from the group consisting of SEQ ID NOs: 13, 15, 17, 19, and 21.

6. The nucleic acid according to claim 1, wherein the nucleotide sequence encoding a fusion protein comprising TMPK or a variant thereof comprises a nucleotide sequence encoding a variant of TMPK selected from the group consisting of SEQ ID NOs: 25, 28, 29, and 37.

7. The nucleic acid according to claim 1, wherein the nucleotide sequence encoding a fusion protein comprising TMPK or a variant thereof and CD19 or a variant thereof comprises SEQ ID NO: 5, 42, or 43.

8. The nucleic acid according to claim 1, wherein the nucleotide sequence encoding PD-L1 comprises SEQ ID NO: 2 or 3.

9. The nucleic acid according to claim 1, wherein the nucleotide sequence encoding PD-L1 and the nucleotide sequence encoding a fusion protein comprising TMPK or a variant thereof and CD19 or a variant thereof comprises SEQ ID NO: 6.

10. A nucleic acid comprising a nucleotide sequence that is at least 90% identical to the nucleic acid according to claim 1.

11. The nucleic acid according to claim 1, wherein the nucleotide sequence encoding programmed death ligand-1 (PD-L1) encodes SEQ ID NO: 1 and the nucleotide sequence encoding a fusion protein comprising thymidylate kinase (TMPK) or a variant thereof and CD19 or a variant thereof encodes SEQ ID NO: 4, 41, or 44.

12. A recombinant expression vector comprising the nucleic acid according to claim 1.

13. The recombinant expression vector according to claim 12, wherein the recombinant expression vector is a lentiviral vector.

14. The recombinant expression vector according to claim 13, wherein the lentiviral vector comprises SEQ ID NO: 7.

15. A host cell comprising the recombinant expression vector of claim 12.

16. The host cell according to claim 15, wherein the host cell is rapamycin-resistant.

17. A population of cells comprising at least one host cell of claim 15.

18. The population of cells according to claim 17, wherein the population of cells is rapamycin-resistant.

19. A pharmaceutical composition comprising the nucleic acid of claim 1, and a pharmaceutically acceptable carrier.

20-24. (canceled)

25. A kit comprising the nucleic acid of claim 1, and a prodrug.

26. The kit according to claim 25, wherein the prodrug is AZT.

27. The kit according to claim 25, further comprising at least one T cell.

28. The kit according to claim 27, wherein the T cell has antigenic specificity for a bacterial antigen, a viral antigen, a tumor antigen, a fungal antigen, or a parasitic antigen.

29. The kit according to claim 25, further comprising a SHP1/2 inhibitor.

30. The kit according to claim 29, wherein the SHP1/2 inhibitor is pervanadate or NSC87877.

31-33. (canceled)

34. A method of treating or preventing a disease in a host comprising administering to the host the nucleic acid of claim 1 in an amount effective to treat or prevent the disease in the host.

35. The method according to claim 34, wherein the disease is an autoimmune disease or an allo-immune disease.

36. A method of suppressing an immune response in a host comprising administering to the host the nucleic acid of claim 1 in an amount effective to suppress the immune response in the host.

37. The method according to claim 34, further comprising administering a prodrug to the host.

38. The method according to claim 37, wherein the prodrug is AZT.

39. The method according to claim 34, further comprising administering to the host at least one SHP1/2 inhibitor-treated T cell(s) in an amount effective to mediate an immune response against a bacterial antigen, a viral antigen, a tumor antigen, a fungal antigen, or a parasitic antigen in the host.

40. The method according to claim 39, wherein the SHP1/2 inhibitor is pervanadate or NSC87877.

41. The method according to claim 39, wherein the SHP1/2 inhibitor-treated T cell(s) have antigenic specificity for a bacterial antigen, a viral antigen, a tumor antigen, a fungal antigen, or a parasitic antigen.


SEQUENCE LISTING (47 sequences)

SEQ ID NO: 1 - protein, 290 aa, Homo Sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 3 - DNA, 873 bp, Artificial Sequence
  Synthetic
atgcggatcttcgccgtgttcattttcatgacctactggcacctgctgaacgccttcaccgtgaccgtgcccaaagatctgtacgtggtggagtacggcagcaacatgacaatcgaatgtaaatttcccgtggagaagcagctggacctggccgccctgatcgtgtactgggagatggaggataagaatatcatccagttcgtgcacggcgaggaggacctgaaagtgcagcactctagctacaggcagcgggcaagactgctcaaggaccagctgagcctggggaacgccgccctccagatcaccgacgtgaaactccaggacgcaggggtgtatcggtgcatgattagctacggcggcgccgactataaacgcatcacagtgaaggtgaacgccccctacaataagatcaaccagagaatcctggtggtggaccccgtgacctctgagcacgaactgacatgccaggccgagggctatccaaaagccgaagtgatctggaccagcagcgaccaccaggtgctgtctgggaagacaacaaccaccaattccaagcgggaggagaagctgttcaacgtgacaagcacactgcggatcaatacaaccaccaacgagatcttctactgtacatttcggcgcctcgatcccgaggagaaccacaccgccgagctggtcatccctgaactgcccctggctcacccaccaaacgagcggacccacctcgtgatcctgggcgccatcctgctgtgcctgggcgtggccctgacctttatctttcggctgagaaagggccggatgatggacgtgaagaaatgcggcatccaggacaccaacagcaagaagcagagcgatacccacctggaagaaacctga

SEQ ID NO: 4 - protein, 529 aa, Artificial Sequence
  Synthetic
MPPPRLLFFLLFLTPMEVRPEEPLVVKVEEGDNAVLQCLKGTSDGPTQQLTWSRESPLKPFLKLSLGLPGLGIHMRPLASWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMGLLLPRATAQDAGKYYCHRGNLTMSFHLEITARPVLWHWLLRTGGWKVSAVTLAYLIFCLCSLVGILHLAGGAAGMAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIATATEKPLGELWK

SEQ ID NO: 5 - DNA, 1596 bp, Artificial Sequence
  Synthetic
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttgccggcggggctgcagggatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 6 - DNA, 3056 bp, Artificial Sequence
  Synthetic
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttgccggcggggctgcagggatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatctcgattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgattctagaatgcggatcttcgccgtgttcattttcatgacctactggcacctgctgaacgccttcaccgtgaccgtgcccaaagatctgtacgtggtggagtacggcagcaacatgacaatcgaatgtaaatttcccgtggagaagcagctggacctggccgccctgatcgtgtactgggagatggaggataagaatatcatccagttcgtgcacggcgaggaggacctgaaagtgcagcactctagctacaggcagcgggcaagactgctcaaggaccagctgagcctggggaacgccgccctccagatcaccgacgtgaaactccaggacgcaggggtgtatcggtgcatgattagctacggcggcgccgactataaacgcatcacagtgaaggtgaacgccccctacaataagatcaaccagagaatcctggtggtggaccccgtgacctctgagcacgaactgacatgccaggccgagggctatccaaaagccgaagtgatctggaccagcagcgaccaccaggtgctgtctgggaagacaacaaccaccaattccaagcgggaggagaagctgttcaacgtgacaagcacactgcggatcaatacaaccaccaacgagatcttctactgtacatttcggcgcctcgatcccgaggagaaccacaccgccgagctggtcatccctgaactgcccctggctcacccaccaaacgagcggacccacctcgtgatcctgggcgccatcctgctgtgcctgggcgtggccctgacctttatctttcggctgagaaagggccggatgatggacgtgaagaaatgcggcatccaggacaccaacagcaagaagcagagcgatacccacctggaagaaacctga

SEQ ID NO: 7 - DNA, 10520 bp, Artificial Sequence
  Synthetic
gggcgaattgggcccgacgtcgcatgcttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacgggatcgattttaaaagaaaaggggggattggggggtacagtgcaggggaaagaatagtagacataatagcaacagacatacaaactaaagaattacaaaaacaaattacaaaaattcaaaattttatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttgccggcggggctgcagggatggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtgaggatctcgattccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgattctagaatgcggatcttcgccgtgttcattttcatgacctactggcacctgctgaacgccttcaccgtgaccgtgcccaaagatctgtacgtggtggagtacggcagcaacatgacaatcgaatgtaaatttcccgtggagaagcagctggacctggccgccctgatcgtgtactgggagatggaggataagaatatcatccagttcgtgcacggcgaggaggacctgaaagtgcagcactctagctacaggcagcgggcaagactgctcaaggaccagctgagcctggggaacgccgccctccagatcaccgacgtgaaactccaggacgcaggggtgtatcggtgcatgattagctacggcggcgccgactataaacgcatcacagtgaaggtgaacgccccctacaataagatcaaccagagaatcctggtggtggaccccgtgacctctgagcacgaactgacatgccaggccgagggctatccaaaagccgaagtgatctggaccagcagcgaccaccaggtgctgtctgggaagacaacaaccaccaattccaagcgggaggagaagctgttcaacgtgacaagcacactgcggatcaatacaaccaccaacgagatcttctactgtacatttcggcgcctcgatcccgaggagaaccacaccgccgagctggtcatccctgaactgcccctggctcacccaccaaacgagcggacccacctcgtgatcctgggcgccatcctgctgtgcctgggcgtggccctgacctttatctttcggctgagaaagggccggatgatggacgtgaagaaatgcggcatccaggacaccaacagcaagaagcagagcgatacccacctggaagaaacctgaggatccaagcttcaattgtggtcactcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 8 - DNA, 1179 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - DNA, 28 bp, Artificial Sequence
  Synthetic
cacaaattttgtaatccagaggttgatt

SEQ ID NO: 10 - DNA, 23 bp, Artificial Sequence
  Synthetic
ctagggaaaaattctccttggca

SEQ ID NO: 11 - DNA, 19 bp, Artificial Sequence
  Synthetic
acacggcgaagatccgcat

SEQ ID NO: 12 - DNA, 20 bp, Artificial Sequence
  Synthetic
tcctggctgaggctctggtt

SEQ ID NO: 13 - DNA, 639 bp, Homo sapiens
  misc_feature: TMPK
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaagtga

SEQ ID NO: 14 - protein, 212 aa, Homo sapiens
  MISC_FEATURE: TMPK
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 15 - DNA, 639 bp, Homo sapiens; identical to SEQ ID NO: 13
  misc_feature: TMPK

SEQ ID NO: 16 - protein, 212 aa, Homo sapiens; identical to SEQ ID NO: 14
  MISC_FEATURE: TMPK

SEQ ID NO: 17 - DNA, 636 bp, Homo sapiens
  misc_feature: TMPK
atggcggcccggcgcggggctctcatagtgctggagggcgtggaccgcgccgggaagagcacgcagagccgcaagctggtggaagcgctgtcgcgcgggccaccgcccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagactcgaagctgtccatgaggaactccgcgtgctctctgaggacgccatccgcactgccacagagaagccgctgggggagctatggaagtga

SEQ ID NO: 18 - protein, 211 aa, Homo sapiens
  MISC_FEATURE: TMPK
MAARRGALIVLEGVDRAGKSTQSRKLVEALSRGPPPELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKRLEAVHEELRVLSEDAIRTATEKPLGELWK

SEQ ID NO: 19 - DNA, 639 bp, Homo sapiens
  misc_feature: TMPK
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagattggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatccgcactgccacagagaagccgctgggggagctatggaaggac

SEQ ID NO: 20 - protein, 213 aa, Homo sapiens
  MISC_FEATURE: TMPK
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWKD

SEQ ID NO: 21 - DNA, 639 bp, Mus musculus
  misc_feature: TMPK
atggcgtcgcgtcggggagcgctcatcgtgctggagggtgtggaccgtgctggcaagaccacgcagggcctcaagctggtgaccgcgctgtgcgcctcgggccacagagcggagctgctgcgtttccccgaaagatcaacggaaatcggcaagcttctgaattcctacttggaaaagaaaacggaactagaggatcactccgtgcacctgctcttctctgcaaaccgctgggaacaagtaccattaattaaggcgaagttgaaccagggtgtgacccttgttttggacagatacgccttttctggggttgccttcactggtgccaaagagaatttttccctggattggtgtaaacaaccggacgtgggccttcccaaacctgacctgatcctgttccttcagttacaattgctggacgctgctgcacggggagagtttggccttgagcgatatgagaccgggactttccaaaagcaggttctgttgtgtttccagcagctcatggaagagaaaaacctcaactggaaggtggttgatgcttccaaaagcattgaggaagtccataaagaaatccgtgcacactctgaggacgccatccgaaacgctgcacagaggccactgggggagctatggaaataa

SEQ ID NO: 22 - protein, 212 aa, Mus musculus
  MISC_FEATURE: TMPK
MASRRGALIVLEGVDRAGKTTQGLKLVTALCASGHRAELLRFPERSTEIGKLLNSYLEKKTELEDHSVHLLFSANRWEQVPLIKAKLNQGVTLVLDRYAFSGVAFTGAKENFSLDWCKQPDVGLPKPDLILFLQLQLLDAAARGEFGLERYETGTFQKQVLLCFQQLMEEKNLNWKVVDASKSIEEVHKEIRAHSEDAIRNAAQRPLGELWK

SEQ ID NO: 23 - protein, 212 aa, Homo sapiens
  MISC_FEATURE: TMPK variant
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIRTATEKPLGELWK

SEQ ID NO: 24 - protein, 214 aa, Homo sapiens
  MISC_FEATURE: TMPK variant
MAARRGALIVLEGVDGAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLTPEVGLKRARARGELDRYENGAFQERALRCFHQLMKDTTLNWKMVDASKSIEAVHEDIRVLSEDAIATATEKPLGELWK

SEQ ID NO: 25 - DNA, 639 bp, Homo sapiens
  misc_feature: TMPK variant
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 26 - protein, 212 aa, Homo sapiens
  MISC_FEATURE: TMPK variant
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAFTGAKENFSLDWCKQPDVGLPKPDLVLFLQLQLADAAKRGAFGHERYENGAFQERALRCFHQLMKDTTLNWKMVDASKSIEAVHEDIRVLSEDAIATATEKPLGELWK

SEQ ID NO: 27 - DNA, 592 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 28 - DNA, 639 bp, Homo sapiens
  misc_feature: TMPK variant
atggcggcccggcgcggggctctcatagtgctggagggcgtggaccgcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggcctacacaggtgccaaggagaatttttccctagactggtgtaaacagccagacgtgggccttcccaaacccgacctggtcctgttcctccagttacagctggcggatgctgccaagcggggagcgtttggccatgagcgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 29 - DNA, 645 bp, Homo sapiens
  misc_feature: TMPK variant
atggcggcccggcgcggggctctcatagtgctggagggcgtggacggcgccgggaagagcacgcagagccgcaagctggtggaagcgctgtgcgccgcgggccaccgcgccgaactgctccggttcccggaaagatcaactgaaatcggcaaacttctgagttcctacttgcaaaagaaaagtgacgtggaggatcactcggtgcacctgcttttttctgcaaatcgctgggaacaagtgccgttaattaaggaaaagttgagccagggcgtgaccctcgtcgtggacagatacgcattttctggtgtggccttcaccggtgccaaggagaatttttccctagactggtgtaaacagccagacgtgggccttcccaaacccgacctggtcctgttcctgcagttaactccggaagttggcttaaaacgcgcacgtgctcgcggcgagcttgaccgctatgagaacggggctttccaggagcgggcgctccggtgtttccaccagctcatgaaagacacgactttgaactggaagatggtggatgcttccaaaagcatcgaagctgtccatgaggacatccgcgtgctctctgaggacgccatcgccactgccacagagaagccgctgggggagctatggaagtga

SEQ ID NO: 30 - DNA, 1966 bp, Homo sapiens
  misc_feature: CD19
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaaaaaaaaaaaaaaaaaa

SEQ ID NO: 31 - protein, 556 aa, Homo sapiens [sequence omitted]

SEQ ID NO: 32 - protein, 313 aa, Homo sapiens
  MISC_FEATURE: CD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 33 - DNA, 939 bp, Homo sapiens
  misc_feature: CD19
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctt

SEQ ID NO: 34 - protein, 313 aa, Homo sapiens
  MISC_FEATURE: CD19
MPPPRLLFFLLFLTPMEVRPEEPLVVKVEEGDNAVLQCLKGTSDGPTQQLTWSRESPLKPFLKLSLGLPGLGIHMRPLASWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 35 - protein, 6 aa, Artificial Sequence
  Synthetic
AGGAAG

SEQ ID NO: 36 - DNA, 18 bp, Artificial Sequence
  Synthetic
gccggcggggctgcaggg

SEQ ID NO: 37 - DNA, 639 bp, Artificial Sequence; identical to SEQ ID NO: 28
  Synthetic

SEQ ID NO: 38 - DNA, 939 bp, Artificial Sequence
  Synthetic
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggcatcctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctt

SEQ ID NO: 39 - protein, 212 aa, Artificial Sequence
  Synthetic
MAARRGALIVLEGVDRAGKSTQSRKLVEALCAAGHRAELLRFPERSTEIGKLLSSYLQKKSDVEDHSVHLLFSANRWEQVPLIKEKLSQGVTLVVDRYAFSGVAYTGAKENFSLDWCKQPDVGLPKPDLVLFLQLQLADAAKRGAFGHERYENGAFQERALRCFHQLMKDTTLNWKMVDASKSIEAVHEDIRVLSEDAIATATEKPLGELWK

SEQ ID NO: 40 - protein, 313 aa, Artificial Sequence; identical to SEQ ID NO: 34
  Synthetic

SEQ ID NO: 41 - protein, 531 aa, Artificial Sequence [sequence omitted]

SEQ ID NO: 42 - DNA, 1596 bp, Artificial Sequence; identical to SEQ ID NO: 5
  Synthetic

SEQ ID NO: 43 - DNA, 1596 bp, Artificial Sequence; identical to SEQ ID NO: 5
  Synthetic

SEQ ID NO: 44 - protein, 531 aa, Artificial Sequence [sequence omitted]

SEQ ID NO: 45 - DNA, 894 bp, Homo sapiens
  misc_feature: CD20 - Membrane-spanning 4-domains, subfamily A, member 1 (MS4A1)
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 46 - DNA, 819 bp, Homo sapiens
  misc_feature: CD25 - Interleukin 2 Receptor, alpha (IL2RA)
atggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctag

SEQ ID NO: 47 - DNA, 1284 bp, Homo sapiens
  misc_feature: LNGFR - Nerve Growth Factor Receptor
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtga